(19) United States
(12) Patent Application Publication
(10) Pub. No.: US 2022/0189581 A1
(43) Pub. Date: Jun. 16, 2022

(54) METHOD AND APPARATUS FOR CLASSIFICATION AND/OR PRIORITIZATION OF GENETIC VARIANTS

(71) Applicant: Nostos Genomics UG, Berlin (DE)

(72) Inventors: David Alberto NEVILLE, Berlin (DE); Rocio ACUNA-HIDALGO, Berlin (DE)

(21) Appl. No.: 17/117,519

(22) Filed: Dec. 10, 2020

(51) Int. Cl. G16B 25/00; G06N 3/12; G16B 5/00

(52) U.S. Cl. CPC G16B 25/00; G16B 5/00; G06N 3/12

(57) ABSTRACT

A method of classifying a genetic variant comprising receiving, at a first plurality of trained nodes of a hierarchical Bayesian Network, input data comprising data of a genetic variant of a patient, receiving, at a second plurality of trained nodes of the hierarchical Bayesian Network, input data comprising said data, receiving, at one or more trained nodes of the hierarchical Bayesian Network input from the first plurality of trained nodes and from the second plurality of trained nodes, providing by the one or more nodes a posterior probability of a functional disruption the genetic variant causes and classifying the genetic variant using said posterior probability of the functional disruption caused by the genetic variant. The first plurality of nodes within the plurality of trained nodes are configured to represent a constraint of a genetic region to variation and the second plurality of nodes within the plurality trained nodes are configured to represent molecular consequences of a genetic variant.

### FIELD

Embodiments described herein relate generally to methods of classifying and/or prioritizing of genetic variants, models and systems for classifying and/or prioritizing of genetic variants and methods for training such models.

### SEQUENCE LISTING

This application contains a Sequence Listing which is concurrently being submitted electronically in ASCII format and is hereby incorporated by reference in its entirety. Said ASCII copy, created on Mar. 17, 2021, is named MARKS-24_Sequence_List.txt and is 1,121 bytes in size.

### BACKGROUND

Around 300 million people worldwide are affected by genetic diseases, ranging from inherited predisposition to cancer to birth defects. Genetic tests are used to diagnose these conditions, by identifying changes in DNA leading to disease. However, genetic testing identifies such disease-causing genetic variation only in a minority of cases, leaving over 60% of patients suspected to have a genetic disease with negative or unclear genetic test results. This entails that most people with a genetic disorder do not receive a clear diagnosis, needed for appropriate medical treatment and genetic counselling.

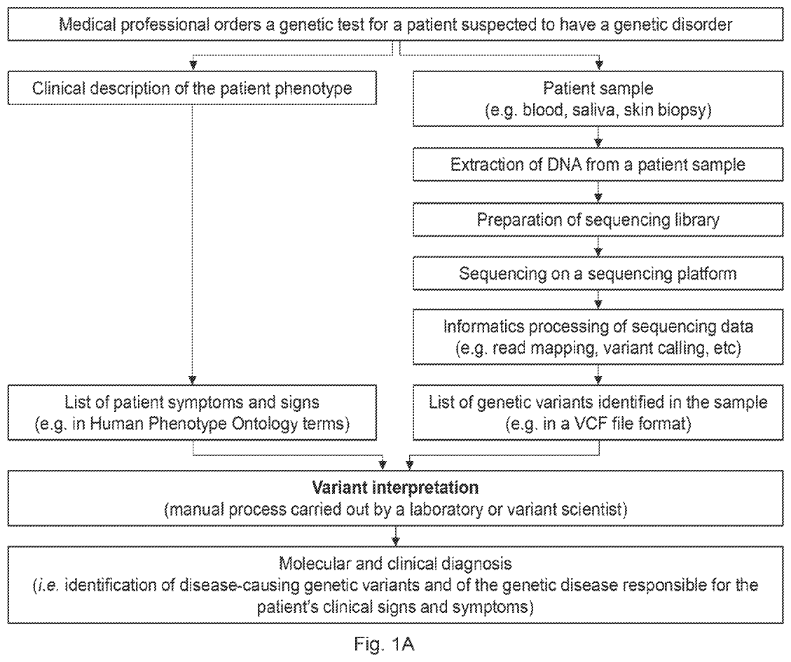

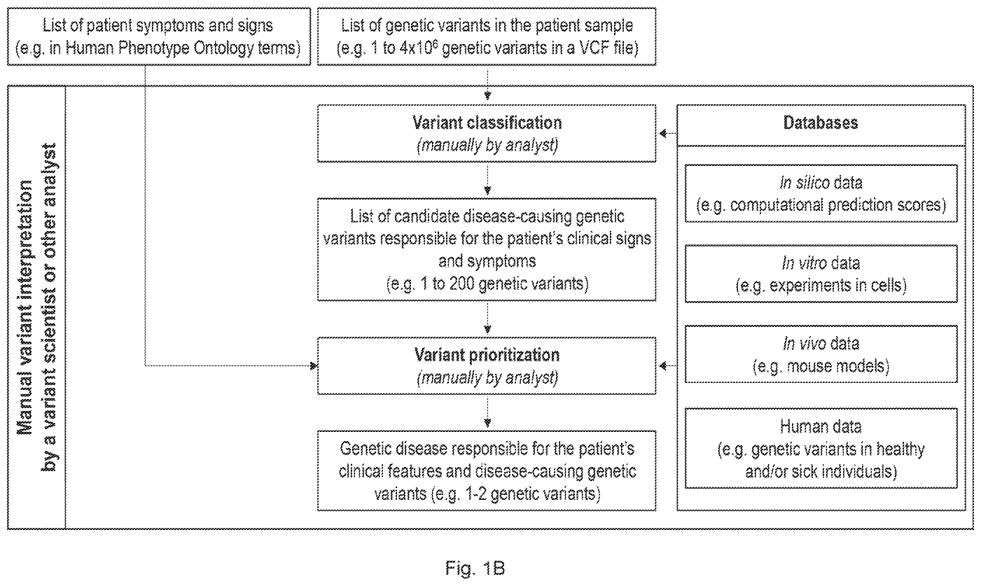

A patient suspected to have a genetic disorder may undergo clinical genetic testing, such as targeted gene sequencing, whole exome sequencing or whole genome sequencing. A description of the patient's clinical signs and symptoms is sent to a clinical genetics laboratory together with a sample of saliva or blood collected from the patient, from which DNA is extracted for testing. The patient's DNA is then processed and sequenced, resulting in a list of genetic variants found in the sample. Genetic variants are differences in the genome (or in the DNA sequence) observed between individuals or populations compared to the reference genome. This includes single nucleotide variants (SNVs), multi-nucleotide variants, insertions, deletions, structural variants and complex rearrangements. The term “genetic variant” is neutral and is applied both to disease-causing and benign variants. This list of genetic variants is analyzed together with the patient's clinical features in a process known as “variant interpretation,” in order to diagnose the patient (i.e., determine whether they have a genetic disease, which genetic disease they have and which are the genetic variants causative for their genetic disease). FIG. 1A illustrates the workflow for clinical genetic testing, including variant interpretation as a necessary step to provide a molecular and clinical diagnosis for patients undergoing clinical genetic testing.

Depending on the type of test performed, the list of genetic variants identified in a sample can range from a dozen to several million variants. As part of variant interpretation, genetic variants found in the sample can be classified in two categories: those that are clinically relevant for the patient (i.e., of importance for the diagnosis and/or medical treatment of the patient) and those that are not. This process is known as “variant classification,” and consists of an initial step where lists of variants identified in a sample are filtered according to some genetic and biological features, followed by individual analysis of candidate genetic variants. In a separate process for variant interpretation, known as “variant prioritization,” the classified candidate genetic variants can be prioritized according to the patient's clinical features and suspected disorder.

Variant interpretation, including variant classification and variant prioritization, is a major challenge and bottleneck in genetic testing, as it consists in identifying correctly few relevant disease-causing genetic variants (usually one or two) among millions of different variants present in the genome of each person. FIG. 1B illustrates the process in which genetic variants are presently interpreted by a human expert (i.e., an analyst), known as a laboratory or variant scientist. This complex process involves integrating data from various sources and is currently done mostly manually, wherein the analyst obtains an annotated VCF file, which contains information about the genetic variants present in a DNA sample taken from a patient, and then performs variant classification and prioritization, themselves based on information that is currently available to them in various databases, such as in silico data, in vitro data, in vivo data and human data. Therefore, this process is time-consuming, expensive and often delivers inaccurate results.

In the following, embodiments will be described with reference to the drawings in which:

FIG. 1A illustrates a current/known process of genetic testing, including variant interpretation;

FIG. 1B illustrates a current/known, manual process of variant interpretation consisting of variant classification and variant prioritization by a human expert;

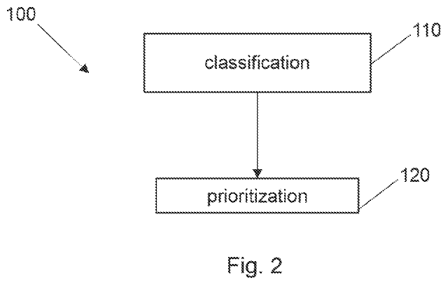

FIG. 2 illustrates an embodiment comprising a genetic variant classification step and a genetic variant prioritization step;

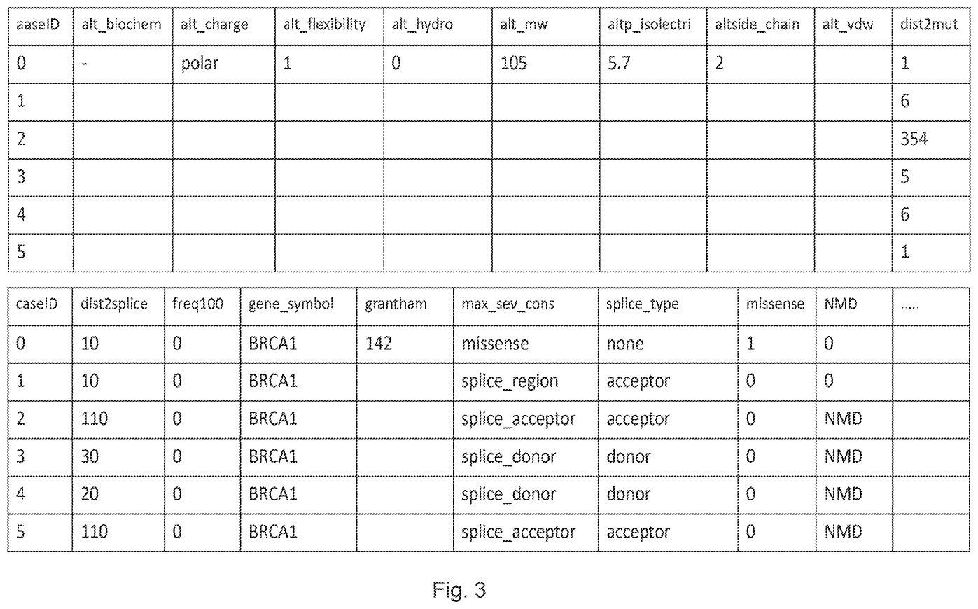

FIG. 3 shows an example of input data for the classification step shown in FIG. 2;

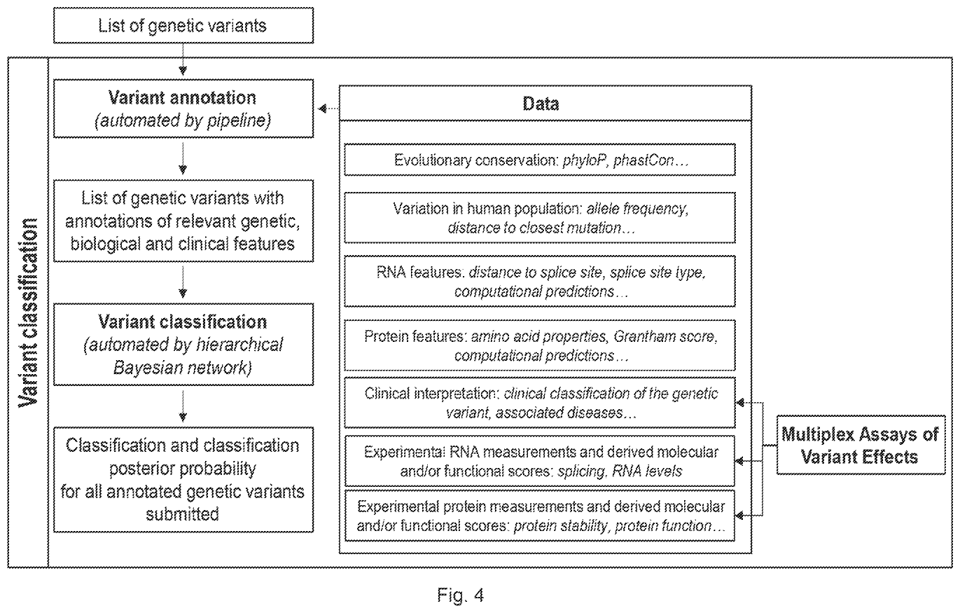

FIG. 4 illustrates an embodiment of the process of automated variant classification shown in FIG. 2, including the data sources that can be used in the variant classification step;

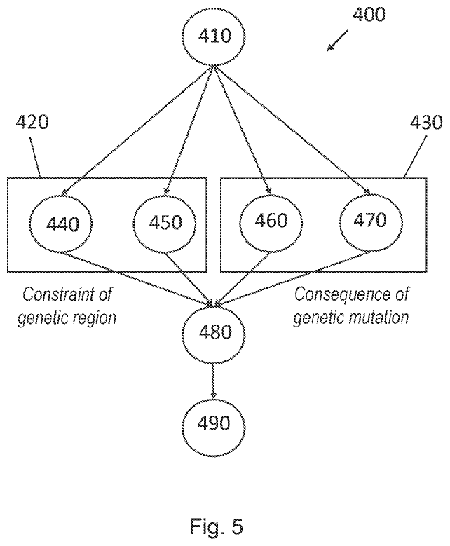

FIG. 5 shows an example of a classification network;

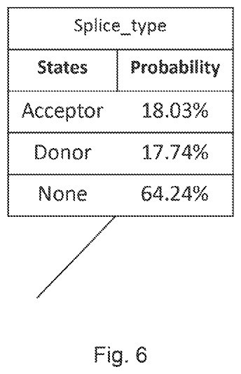

FIG. 6 shows an example of a discrete node that can be used in the network shown in FIG. 5;

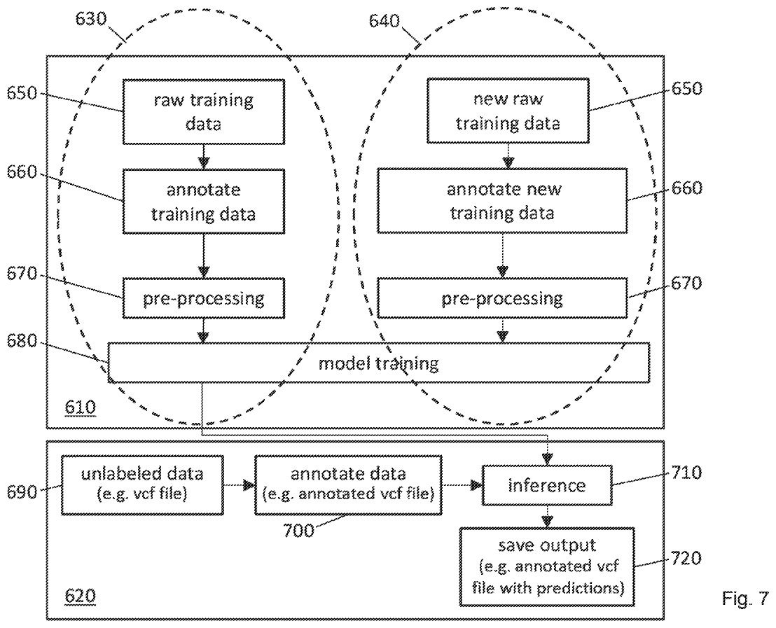

FIG. 7 shows a flowchart illustrating methods of training and use of a hierarchical Bayesian network serving as a genetic variant classifier;

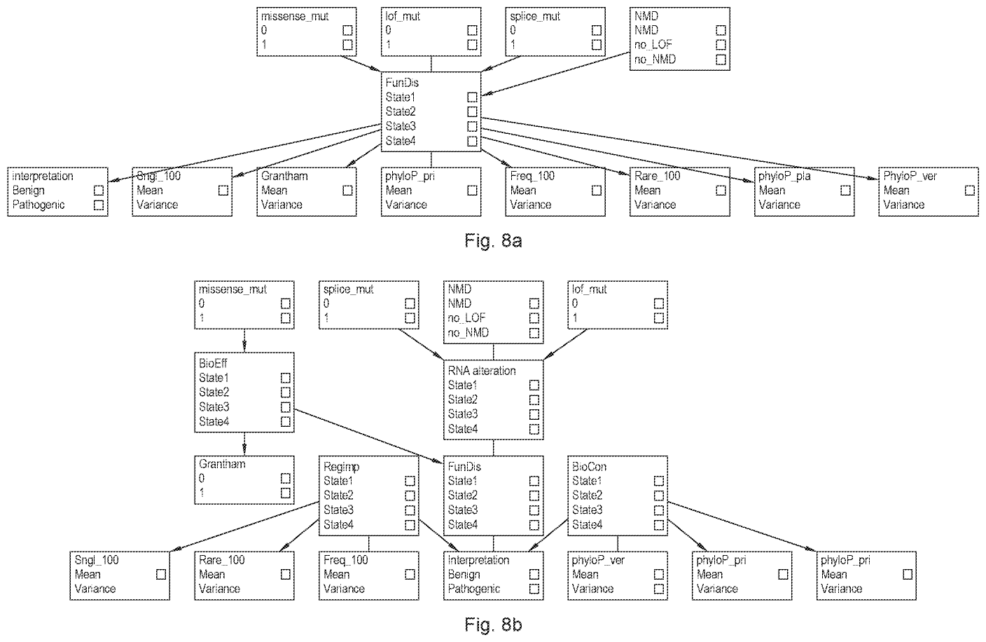

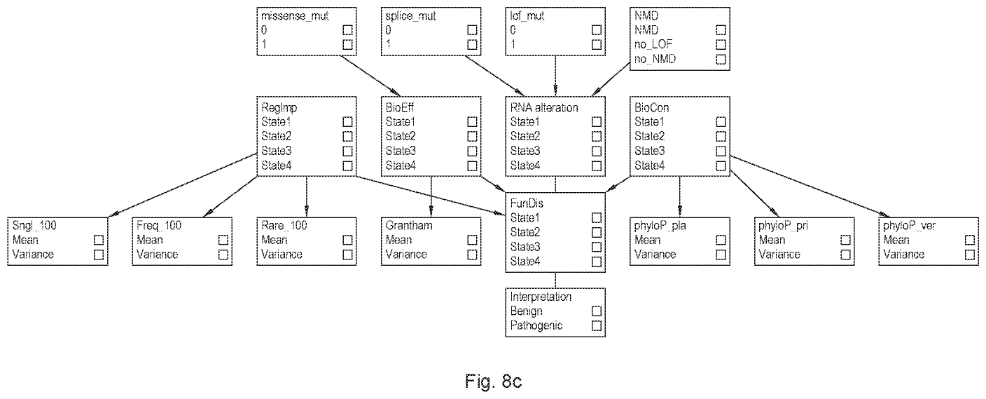

FIG. 8A, illustrates a first variation of a candidate graph structure;

FIG. 8B, illustrates a second variation of a candidate graph structure;

FIG. 8C, illustrates a third variation of a candidate graph structure;

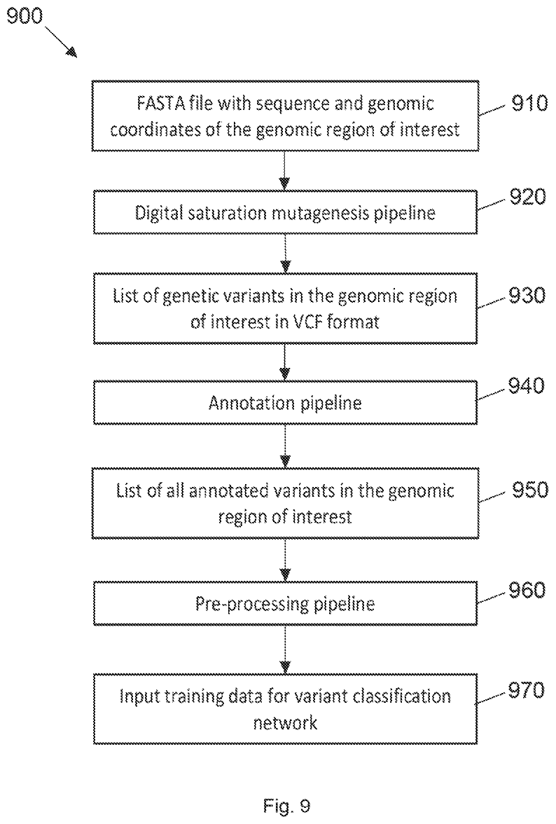

FIG. 9 illustrates a process through which training data for the variant classification network is generated and annotated using a bioinformatics pipeline;

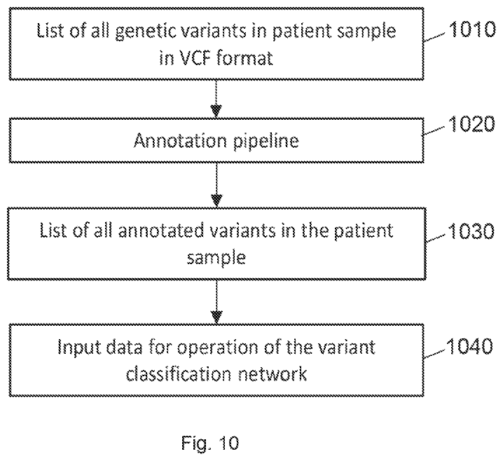

FIG. 10 illustrates a process through which input data for regular use of the trained variant classification network is annotated using a bioinformatics pipeline;

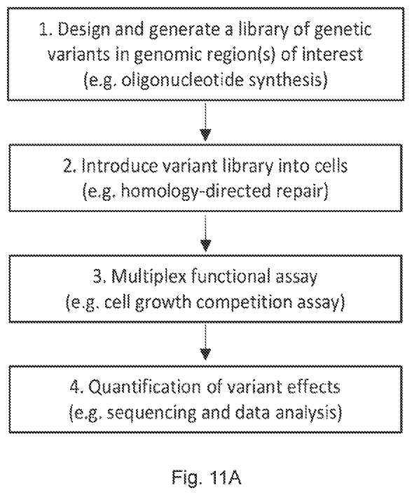

FIG. 11A shows a general experimental outline of a Multiplex Assay of Variant Effects (MAVE);

FIG. 11B illustrates different steps in the general process of performing MAVEs to measure genetic variant effects;

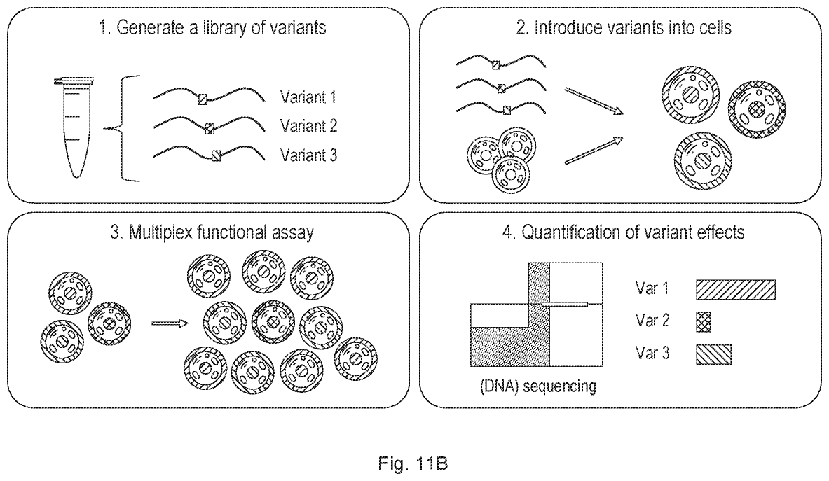

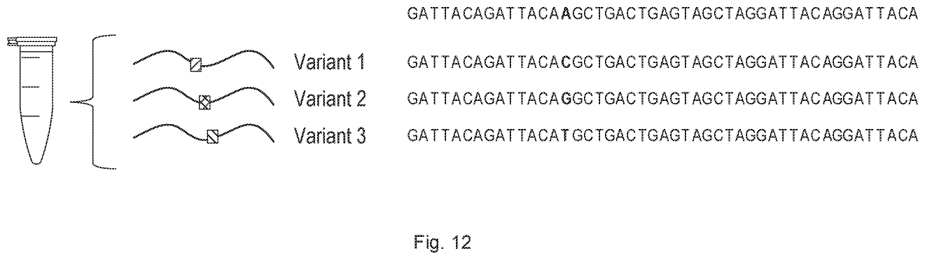

FIG. 12 shows an example of three different oligonucleotide sequences (SEQ ID NOS 1-4 respectively, in order of appearance) designed to generate three different genetic variants at a single base pair position (i.e., saturation mutagenesis of a single base pair), as part of the process of generating variant libraries for mutagenesis[;

FIG. 13 is an illustration of the process through which genetic variants can be introduced into studied cells using homology-directed repair;

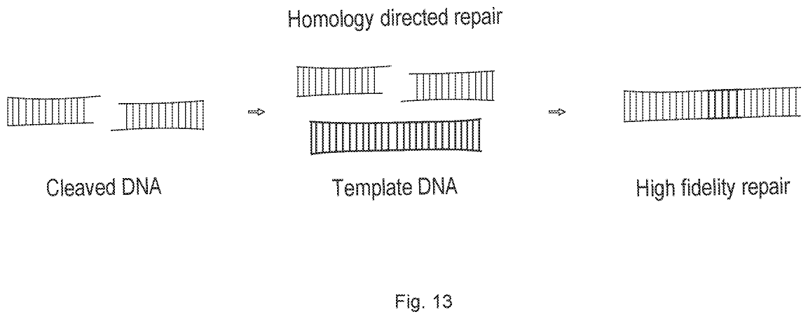

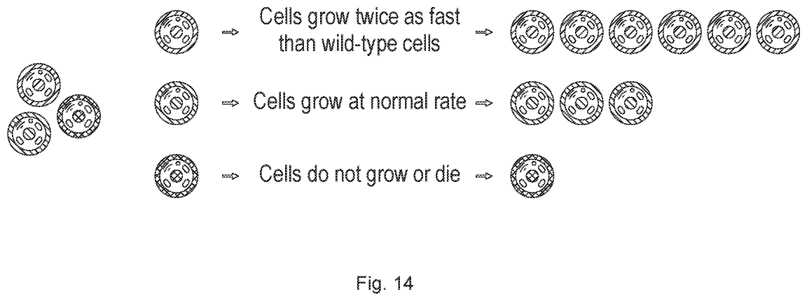

FIG. 14 illustrates competitive cell growth for positive cell selection as an example of a multiplex functional assay to stratify a cell population according to effects of the genetic variants introduced;

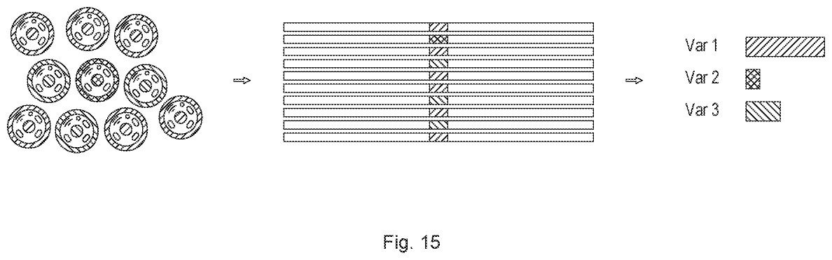

FIG. 15 is an illustration of the quantification of individual genetic variants within a heterogeneous cell population through sequencing and counting the number of sequencing reads with individual genetic variants or variant barcodes as a proxy for the number of cells with individual genetic variants within the population;

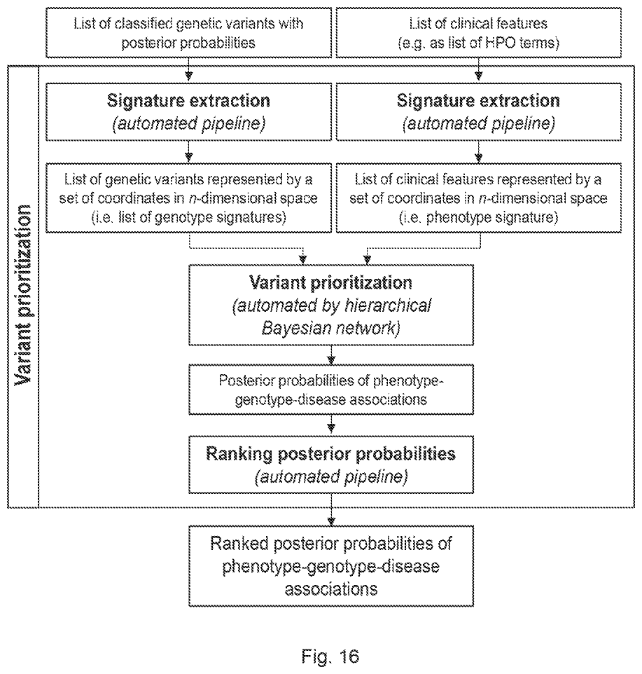

FIG. 16 shows an example of a process of automated variant prioritization shown in FIG. 2:

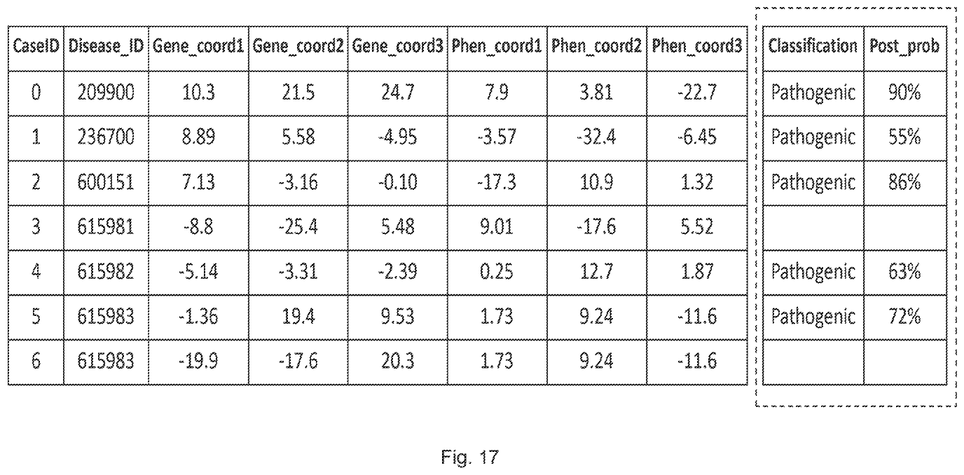

FIG. 17 shows an example of input data for the variant prioritization network;

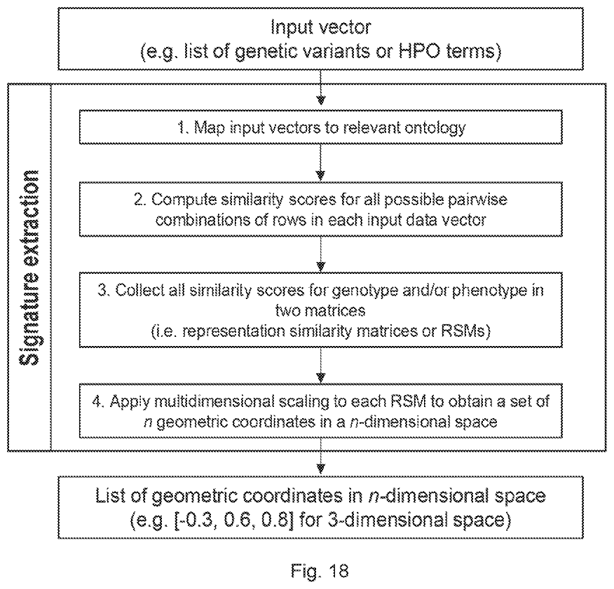

FIG. 18 shows steps of signature extraction for pre-processing of the genetic and phenotype data prior to being input in the variant prioritization network;

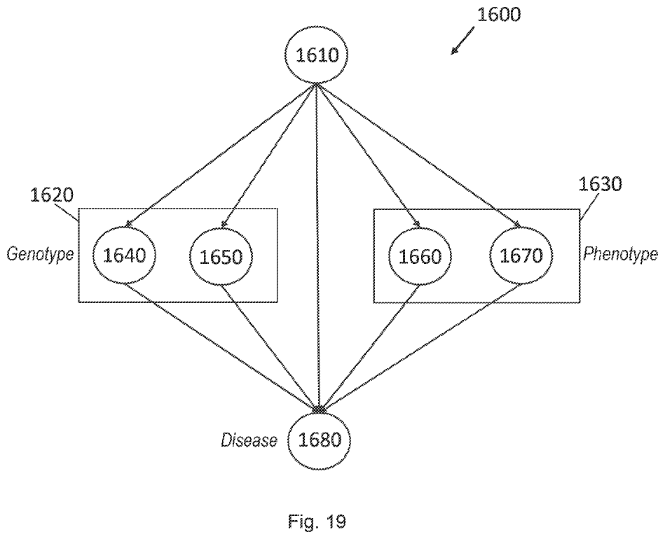

FIG. 19 shows an example of a prioritization network; and

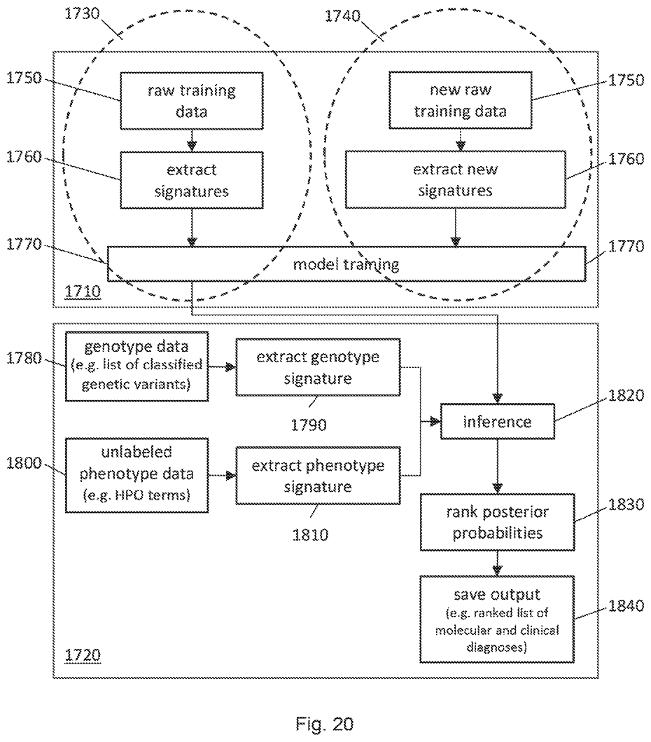

FIG. 20 shows a flowchart illustrating methods of training and use of a hierarchical Bayesian network genetic variant prioritizer of an embodiment.

### DETAILED DESCRIPTION

In an embodiment, there is provided a model for classifying genetic variants. The model comprises a trained hierarchical Bayesian Network comprising a first plurality of trained nodes configured to receive input data comprising data of a genetic variant of a patient, a second plurality of trained nodes configured to receive said input data, one or more nodes configured to receive input from the first plurality of trained nodes and from the second plurality of trained nodes, the one or more nodes configured to provide a posterior probability of a functional disruption the genetic variant causes and to classify the genetic variant using said posterior probability of the functional disruption caused by the genetic variant. The first plurality of nodes within the plurality of trained nodes are configured to represent a constraint of a genetic region to variation. The second plurality of nodes within the plurality trained nodes are configured to represent molecular consequences of a genetic variant.

In one embodiment, the functional disruption the genetic variant causes is expressed in terms of the severity of the functional disruption on the gene or gene product caused by the genetic variant. This may be expressed through categories, such as severe functional disruption, moderate functional disruption, mild functional disruption or no functional disruption. Alternatively, a numerical value within a predetermined range, for example a percentage range from 0% to 100% may be used to express the degree of expected functional disruption.

In one embodiment, the classification output is a measure of the clinical impact expected of the genetic variant. The output may, for example, classify the genetic variant as pathogenic or benign. Alternatively or additionally the classification output may provide an indication of the molecular effect caused by the genetic variant, for example by specifying if the genetic variant is likely to be damaging to the function or structure of a genetic region, gene or gene product or is unlikely to compromise the function or structure of a genetic region, gene or gene product.

In one embodiment, the constraint of the genetic region represented by the first group of trained nodes includes the evolutionary constraint of a gene or genetic region and/or tolerance to variation of a gene or genetic region in human populations.

In one embodiment, the molecular consequences of a genetic variant represented by the second group of trained nodes includes molecular consequences at the RNA and at the protein level.

In an embodiment, the output node is configured to output the classification, optionally alongside a score indicating the probability and confidence in the reliability of the classification.

In an embodiment, at least one of nodes of the first plurality of trained nodes or nodes of the second plurality of trained nodes is further configured to receive experimentally determined input data of at least one of the molecular consequences or the functional consequences of a genetic variant.

By enabling input of experimental data, the model can be trained based on data created in laboratory experiments independent of patient specific genomic variant data. In one embodiment, the experimental data are data generated by high-throughput biological experiments. In a particular embodiment the experiments are Multiplex Assays of Variant Effects.

In one embodiment, the molecular and/or cellular effects of genetic variants determined in laboratory experiments are processed and used to train an intermediate specialized machine learning model, from which predictions for scores are generated. These predicted scores are used, in turn, as a feature in the input data to train the hierarchical Bayesian Network for variant classification.

In one embodiment, the predictions for scores are predictions for molecular or functional scores.

In an embodiment, the input data further comprise at least one of one or more individual genetic features of the genetic variant, one or more biological features of the genetic variant or one or more clinical features of the genetic variant.

In an embodiment, there is provided a system comprising the above described model and an input interface for the model. The input interface is configured to receive the genomic variant data, for example data generated during clinical genetic testing, and to annotate data of individual genomic variants by adding at least one of one or more individual genetic features of the genetic variant, one or more biological features of the genetic variant or one or more clinical features of the genetic variant.

In an embodiment, there is provided a model for ranking a plurality of classified genetic variants comprising a trained hierarchical Bayesian Network comprising a first plurality of trained nodes configured to receive input data comprising an indication of a biological relationship of a genetic variant of the patient with other genetic variants in a reference data set of known disease causing genetic variants, the first plurality of trained nodes configured to determine a likelihood of the biological relationship of the genetic variant with other genetic variants in the reference data set of known disease causing genetic variants occurring. The trained hierarchical Bayesian Network further comprises a second plurality of trained nodes configured to receive input data comprising a plurality of clinical features of the patient in relation to other pluralities of clinical features in a reference data set of known clinical features associated with disease. The second plurality of trained nodes is configured to determine a likelihood of the data comprising the plurality of clinical features of the patient occurring in relation to the reference data set of known clinical features associated with disease. The trained hierarchical Bayesian Network further comprises one or more further nodes configured to receive input from the first plurality of trained nodes and from the second plurality of trained nodes. The one or more further nodes configured to at least one of attribute a disease label to the received input data or determine an indication of a likelihood of co-occurrence of the genetic variant of the patient and the plurality of clinical features of the patient.

In an embodiment, the plurality of clinical features includes one or more or all of a phenotype of the patient, including but not limited to observed disease signs and symptoms experienced by the patient, results of laboratory test, imaging results, etc.

In an embodiment, the first plurality of trained nodes is trained to determine the relationship of a genetic variant to other known disease-causing genetic variants in the reference data set. It will be appreciated that the training received by the node is not specific to individual patients and instead relies on a large number of training sets that encode various combinations of genetic variants.

In an embodiment, the second plurality of trained nodes is trained to determine the relationship of a list of clinical features to other lists of clinical features known to be associated with specific genetic diseases (as provided in a reference data set). It will be appreciated that the training received by the node is not specific to individual patients and instead relies on a large number of training sets that encode various combinations of pluralities of clinical features.

In an embodiment, the one or more further nodes is trained to attribute the disease label and/or determine the indication of likelihood of co-occurrence for data relating to individuals. It will be appreciated that the training received by the one or more further nodes is not specific to individual patients and instead relies on a large number of training sets that encode various combinations of genetic variants classified as disease causing in known genetic diseases and their corresponding clinical features. It will moreover be appreciated that such training can but does not have to include a label of a known disease.

In an embodiment, the one or more further nodes are configured to both attribute a disease label to the received input data and determine a likelihood of co-occurrence of the genetic variant of the patient and the phenotype of the patient, the one or more further nodes are configured to receive a plurality of disease labels attributed to a plurality of genetic variants co-occurring with the phenotype of the patient and rank the plurality of disease labels in order of their associated indications of likelihood of co-occurrence of the genetic variant with the phenotype of the patient.

Input data provided to the model for ranking may comprise genetic variants classified by the above-described model for classifying genetic variants.

The input data may further comprise a score indicating a confidence in the reliability of each classification.

In an embodiment, there is provided a system comprising a model as described above and an annotation pipeline configured to receive a plurality of sets of input data. Each set of input data identifies a genetic variant of a patient or a genetic variant classified as disease causing in a known genetic disease, the annotation pipeline configured to generate a representation of each genetic variant by mapping each set of input data to an ontology graph, to determine, for each of a plurality of pairings of two genetic variants, a measure of similarity between the representations of the two genetic variants in the pairing and to determine a signature of each genetic variant by reducing, in a metric preserving manner, the dimensionality of a matrix comprising all measures of similarity relating to the genetic variant.

In an embodiment, there is provided a system comprising a model as described above and an annotation pipeline configured to receive data identifying a plurality of clinical features of a patient or of a known genetic disease, determine, for each of the pairings of a plurality of clinical features, a measure of similarity between pairs of pluralities of clinical features and to determine a signature of each plurality of clinical features by reducing, in a metric preserving manner, the dimensionality of a matrix comprising all measures of similarity relating to the plurality of clinical features.

In another embodiment, there is provided a system comprising a model for classifying genetic variants and a model for ranking a plurality of classified genetic variants. The model for classifying genetic variants comprises a first trained hierarchical Bayesian Network comprising a first plurality of trained nodes configured to receive input data comprising data of a genetic variant of a patient, a second plurality of trained nodes configured to receive said input data and one or more nodes configured to receive input from the first plurality of trained nodes and from the second plurality of trained nodes. The one or more nodes are configured to provide a posterior probability of a functional disruption the genetic variant causes and to classify the genetic variant using said posterior probability of the functional disruption caused by the genetic variant, thereby providing a classified genetic variant. The first plurality of nodes within the plurality of trained nodes are configured to represent a constraint of a genetic region to variation. The second plurality of nodes within the plurality of trained nodes are configured to represent molecular consequences of a genetic variant. The model for ranking the plurality of classified genetic variants comprises a second trained hierarchical Bayesian Network comprising a third plurality of trained nodes configured to receive input data comprising an indication of a biological relationship of the classified genetic variant of the patient with other genetic variants classified in a reference data set of known disease causing genetic variants. The third plurality of trained nodes is configured to determine a likelihood of the biological relationship of the classified genetic variant with other classified genetic variants in the reference data set of known disease-causing genetic variants occurring. The model for ranking the plurality of classified genetic variants further comprises a fourth plurality of trained nodes configured to receive input data comprising a plurality of clinical features of the patient in relation to other pluralities of clinical features in a reference data set of known clinical features associated with disease. The fourth plurality of trained nodes is configured to determine a likelihood of the data comprising the plurality of clinical features of the patient occurring in relation to the reference data set of known clinical features associated with disease. The model for ranking the plurality of classified genetic variants further comprises one or more further nodes configured to receive input from the third plurality of trained nodes and from the fourth plurality of trained nodes. The one or more further nodes configured to at least one of attribute a disease label to the received input data or determine an indication of a likelihood of co-occurrence of the genetic variant of the patient and the plurality of clinical features of the patient.

In an embodiment, there is provided a method of classifying a genetic variant comprising receiving, at a first plurality of trained nodes of a hierarchical Bayesian Network, input data comprising data of a genetic variant of a patient, receiving, said input data at a second plurality of trained nodes of the hierarchical Bayesian Network, receiving, at one or more trained nodes of the hierarchical Bayesian Network input from the first plurality of trained nodes and from the second plurality of trained nodes, providing by the one or more nodes a posterior probability of a functional disruption the genetic variant causes and classifying the genetic variant using said posterior probability of the functional disruption caused by the genetic variant. The first plurality of nodes within the plurality of trained nodes are configured to represent a constraint of a genetic region to variation and wherein the second plurality of nodes within the plurality of trained nodes are configured to represent molecular consequences of a genetic variant.

In an embodiment, there is provided a method comprising receiving input data comprising an indication of a biological relationship of a genetic variant of the patient with other genetic variants in a reference data set of known disease causing genetic variants at a first plurality of trained nodes of a hierarchical Bayesian Network, using the first plurality of trained nodes to determine a likelihood of the biological relationship of the genetic variant with other genetic variants in the reference data set of known disease causing genetic variants occurring, receiving input data comprising a plurality of clinical features of the patient in relation to other pluralities of clinical features in a reference data set of known clinical features associated with disease at a second plurality of trained nodes of a hierarchical Bayesian Network, using the second plurality of trained nodes to determine a likelihood of the data comprising the plurality of clinical features of the patient occurring in relation to the reference data set of known clinical features associated with disease, at least one of attributing a disease label to the received input data or determining an indication of a likelihood of co-occurrence of the genetic variant of the patient and the plurality of clinical features of the patient and using one or more further nodes based on input from the first plurality of trained nodes and from the second plurality of trained nodes.

In an embodiment, there is provided a method comprising receiving, at a first plurality of trained nodes of a first hierarchical Bayesian Network, input data comprising data of a genetic variant of a patient, receiving said input data at a second plurality of trained nodes of the first hierarchical Bayesian Network, receiving, at one or more trained nodes of the first hierarchical Bayesian Network input from the first plurality of trained nodes and from the second plurality of trained nodes, providing by the one or more nodes a posterior probability of a functional disruption the genetic variant causes, providing a classified genetic variant by classifying the genetic variant using said posterior probability of the functional disruption caused by the genetic variant, wherein the first plurality of nodes within the plurality of trained nodes are configured to represent a constraint of a genetic region to variation and wherein the second plurality of nodes within the plurality of trained nodes are configured to represent molecular consequences of a genetic variant, receiving input data comprising an indication of a biological relationship of the classified genetic variant of the patient with other genetic variants in a reference data set of known disease causing genetic variants at a third plurality of trained nodes of a second hierarchical Bayesian Network, using the third plurality of trained nodes to determine a likelihood of the biological relationship of the genetic variant with other genetic variants in the reference data set of known disease causing genetic variants occurring, receiving input data comprising a plurality of clinical features of the patient in relation to other pluralities of clinical features in a reference data set of known clinical features associated with disease at a fourth plurality of trained nodes of the second hierarchical Bayesian Network, using the fourth plurality of trained nodes to determine a likelihood of the data comprising the plurality of clinical features of the patient occurring in relation to the reference data set of known clinical features associated with disease, at least one of attributing a disease label to the received input data or determining an indication of a likelihood of co-occurrence of the genetic variant of the patient and the plurality of clinical features of the patient and using one or more further nodes of the second hierarchical Bayesian Network based on input from the third plurality of trained nodes and from the fourth plurality of trained nodes.

In an embodiment, there are provided computer program instructions for execution by one or more processors, the instructions, when executed by the one or more processors causing the one or more processors to receive, at a first plurality of trained nodes of a hierarchical Bayesian Network, input data comprising data of a genetic variant of a patient, receive said data at a second plurality of trained nodes of the hierarchical Bayesian Network, receive, at one or more trained nodes of the hierarchical Bayesian Network input from the first plurality of trained nodes and from the second plurality of trained nodes, provide by the one or more nodes a posterior probability of a functional disruption the genetic variant causes and classify the genetic variant using said posterior probability of the functional disruption caused by the genetic variant. The first plurality of nodes within the plurality of trained nodes are configured to represent a constraint of a genetic region to variation and wherein the second plurality of nodes within the plurality of trained nodes are configured to represent molecular consequences of a genetic variant.

In an embodiment, there are provided computer program instructions for execution by one or more processors, the instructions, when executed by the one or more processors causing the one or more processors to receive input data comprising an indication of a biological relationship of a genetic variant of the patient with other genetic variants in a reference data set of known disease causing genetic variants at a first plurality of trained nodes of a hierarchical Bayesian Network, use the first plurality of trained nodes to determine a likelihood of the biological relationship of the genetic variant with other genetic variants in the reference data set of known disease causing genetic variants occurring, receive input data comprising a plurality of clinical features of the patient in relation to other pluralities of clinical features in a reference data set of known clinical features associated with disease at a second plurality of trained nodes of a hierarchical Bayesian Network, use the second plurality of trained nodes to determine a likelihood of the data comprising the plurality of clinical features of the patient occurring in relation to the reference data set of known clinical features associated with disease, at least one of attribute a disease label to the received input data or determine an indication of a likelihood of co-occurrence of the genetic variant of the patient and the plurality of clinical features of the patient and use one or more further nodes based on input from the first plurality of trained nodes and from the second plurality of trained nodes.

Computer program instructions for execution by one or more processors, the instructions, when executed by the one or more processors causing the one or more processors to receive, at a first plurality of trained nodes of a first hierarchical Bayesian Network, input data comprising data of a genetic variant of a patient, receive said input data at a second plurality of trained nodes of the first hierarchical Bayesian Network, receive, at one or more trained nodes of the first hierarchical Bayesian Network input from the first plurality of trained nodes and from the second plurality of trained nodes, provide by the one or more nodes a posterior probability of a functional disruption the genetic variant causes, provide a classified genetic variant by classifying the genetic variant using said posterior probability of the functional disruption caused by the genetic variant, wherein the first plurality of nodes within the plurality of trained nodes output a representation of a constraint of a genetic region to variation and wherein the second plurality of nodes within the plurality of trained nodes output a representation of molecular consequences of a genetic variant, receive input data comprising an indication of a biological relationship of the classified genetic variant of the patient with other genetic variants in a reference data set of known disease causing genetic variants at a third plurality of trained nodes of a second hierarchical Bayesian Network, use the third plurality of trained nodes to determine a likelihood of the biological relationship of the genetic variant with other genetic variants in the reference data set of known disease causing genetic variants occurring, receive input data comprising a plurality of clinical features of the patient in relation to other pluralities of clinical features in a reference data set of known clinical features associated with disease at a fourth plurality of trained nodes of the second hierarchical Bayesian Network, use the fourth plurality of trained nodes to determine a likelihood of the data comprising the plurality of clinical features of the patient occurring in relation to the reference data set of known clinical features associated with disease, at least one of attribute a disease label to the received input data or determine an indication of a likelihood of co-occurrence of the genetic variant of the patient and the plurality of clinical features of the patient and use one or more further nodes of the second hierarchical Bayesian Network based on input from the third plurality of trained nodes and from the fourth plurality of trained nodes.

In an embodiment, there is provided a method of training a hierarchical Bayesian Network for classifying genetic variants comprising providing a plurality of candidate network structures, training each candidate network structure of the plurality of candidate network structures using a same data set to create a plurality of trained candidate network structures, creating one or more modified candidate network structures by modifying one or more candidate network structures of the plurality of candidate network structures to increase a predictive performance of the one or more candidate network structures, training each modified candidate network structure of the plurality of modified candidate network structures using a same data set to create a plurality of trained modified candidate network structures and selecting the trained modified candidate network structure of the plurality of trained modified candidate network structures that has a best predictive performance.

In an embodiment, the candidate network structures are originally provided or at least modified based on scientific knowledge of the genomic generative process. In one embodiment, this knowledge may be provided by a human expert.

In an embodiment, the candidate network structures are modified by modifying or pruning one or more connections between nodes in the candidate network structures to emphasise or de-emphasise the one or more connections between the nodes in the candidate network structure.

In one embodiment, the candidate network structures are based on a common original network structure that has been modified or pruned to emphasise or de-emphasise one or more connections between nodes in the original network structure. It will be appreciated that, for different candidate network structure different connections of the original network structure are emphasised or de-emphasised.

In an embodiment, there is provided a method of training a hierarchical Bayesian Network for classifying genetic variants comprising identifying one or more nodes that provide a low predictive quality within the hierarchical Bayesian Network, using biological experiments to generate training data relating to the identified one or more nodes or relating to an observed node providing input to the identified one or more nodes and training said hierarchical Bayesian Network using said training data.

It will be appreciated that the hierarchical Bayesian Network may already be partially trained before the above discussed steps are performed.

In an embodiment, the method further comprises using the data generated using biological experiments to determine at least one of molecular scores or functional scores and training the model using said determined scores.

In an embodiment, there is provided a method of training a hierarchical Bayesian Network for ranking a plurality of classified genetic variants comprising providing a hierarchical Bayesian Network that comprises a first plurality of nodes and a second plurality of nodes, one or more further nodes configured to receive input from the first plurality of nodes and from the second plurality of nodes. The method further comprises training the hierarchical Bayesian Network based on at least one of input data applied to the first plurality of nodes, the input data applied to the first plurality of nodes comprising an indication of a biological relationship of the genetic variant of the patient with other genetic variants in the reference data set of known disease causing genetic variants, the first plurality of nodes outputting a likelihood of the biological relationship of the genetic variant with other genetic variants in the reference data set of known disease causing genetic variants occurring, input data applied to the second plurality of nodes, the input data applied to the second plurality of nodes comprising a plurality of clinical features of the patient in relation to other pluralities of clinical features in a reference data set of known clinical features associated with disease, the second plurality of trained nodes outputting a likelihood of the data comprising the plurality of clinical features occurring in relation to the reference data set of known clinical features associated with disease or a disease label associated with the genetic variant and provided to the one or more further nodes.

In an embodiment, there are provided computer program instructions for execution by one or more processors, the instructions, when executed by the one or more processors causing the one or more processors to train each of a plurality of candidate network structures using a same data set to create a plurality of trained candidate network structures, create one or more modified candidate network structures by modifying one or more candidate network structures of the plurality of candidate network structures to increase a predictive performance of the one or more candidate network structures, training each modified candidate network structure of the plurality of modified candidate network structures using a same data set to create a plurality of trained modified candidate network structures and select the trained modified candidate network structure of the plurality of trained modified candidate network structures that has a best predictive performance.

In an embodiment, there are provided computer program instructions for execution by one or more processors, the instructions, when executed by the one or more processors causing the one or more processors to identify, within a hierarchical Bayesian Network, one or more nodes that provide a low predictive quality, use training data generated using biological experiments relating to the identified one or more nodes or relating to an observed node providing input to the identified one or more nodes to train said hierarchical Bayesian Network.

In an embodiment, there is provided a method of training a hierarchical Bayesian Network for ranking a plurality of classified genetic variants. The method comprises providing a hierarchical Bayesian Network comprising a first plurality of nodes and a second plurality of nodes and one or more further nodes configured to receive input from the first plurality of nodes and from the second plurality of nodes, training the hierarchical Bayesian Network based on at least one of input data applied to the first plurality of nodes, the input data applied to the first plurality of nodes comprising an indication of a biological relationship of a genetic variant of the patient with other genetic variants in a reference data set of known disease causing genetic variants the first plurality of nodes outputting a likelihood of the biological relationship of the genetic variant with other genetic variants in the reference data set of known disease causing genetic variants occurring, input data applied to the second plurality of nodes, the input data applied to the second plurality of nodes comprising a plurality of clinical features of the patient in relation to other pluralities of clinical features in a reference data set of known clinical features associated with disease, the second plurality of nodes outputting a likelihood of the data comprising the plurality of clinical features of the patient occurring in relation to the reference data set of known clinical features associated with disease or a disease label associated with the genetic variant and provided to the one or more further nodes.

Disclosed is a method of automatic variant classification and/or prioritization. When practiced in combination, the disclosed method is consequently a method of variant interpretation. In one embodiment, the disclosed method combines genomics and machine learning. In an embodiment, a decision-support tool and method for variant scientists (i.e., analysts) interpreting genetic data in laboratories performing genetic testing is provided. As shown in FIG. 2, in some embodiments, the decision support method 100 performs two steps, a step 110 of classifying genetic variants according to their effect and a step 120 of prioritizing the classified variants to identify those variants that are most likely to cause a patient's genetic disorder, preferably according to the patient's clinical phenotype. Whilst these steps are shown in FIG. 2 as being performed sequentially, this is not to be understood as meaning that both steps are required to be performed by the same computing entity or device or indeed in the same physical location or by the same virtualized entity. Indeed, in other embodiments, only one of the two steps 110 and 120 may be performed according to the present disclosure, with the other step being performed in a known fashion.

In an embodiment, two different machine learning algorithms are used. In one embodiment, a first classification algorithm is used in step 110 to classify genetic variants as “pathogenic” (i.e., disease-causing or otherwise clinically relevant) or “benign” (i.e., not clinically relevant). In another embodiment, the first classification algorithm is used in step 110 to classify genetic variants as “damaging” (i.e., damaging to a genetic region, gene or gene product such as RNA or protein) or “tolerated” (i.e., without molecular consequence to a genetic region, gene or gene product such as RNA or protein). In another embodiment, the first classification algorithm used in step 110 classifies genetic variants as “pathogenic” or “benign” and additionally as “damaging” or “tolerated.”

The process of giving a classification to a genetic variant is known as variant classification. Variant classification is carried out by a machine learning algorithm which, given the input data, calculates the posterior probability that the variant is pathogenic versus the probability that it is benign. This probability is a numerical value ranging from 0 to 100% incorporating graded measures of the evidence available for the pathogenicity of a given genetic variant. For instance, a variant with a probability of 100% pathogenic/0% benign would indicate a variant for which the evidence provided entirely supports classification of the variant as “pathogenic.” A probability of 80% pathogenic/20% benign would indicate instead that the evidence provided mostly, but not entirely, supports classification as “pathogenic.” The continuity of the probabilities obtained from the machine learning model allows for the classification of genetic variants on a continuous scale (from benign to pathogenic) rather than on a discrete binary scale (either benign or pathogenic). One core advantage of this is the possibility to identify and characterize with great precision cases for which the evidence provided supports a mixture of benign and pathogenic effects. The classification algorithm is trained prior to its use and/or during its use on an ongoing basis.

The input for the process 100 is a list of genetic variants identified during genetic testing (e.g., a VCF file generated by targeted gene sequencing, whole exome sequencing or whole genome sequencing) and a list of clinical features presented by the patient encoded in any ontology that describes clinical phenotypes, such as, for example, the Human Phenotype Ontology (HPO). The output is: 1) a list of genetic variants used as input, individually classified as “benign” or “pathogenic” together with the posterior probability of the classification prediction; and 2) a list of possible genetic diagnoses for the patient together with the probability associated with each diagnosis, given combinations of genetic variants and clinical features observed in the patient. To identify the most likely genetic diagnosis for the patient analyzed, the latter list can be ranked by the posterior probability associated with each individual diagnosis.

In this respect, it is important to note that the use of statistical methods from the Bayesian framework does not by itself constitute the use of Bayesian networks.

Both algorithms for variant classification and prioritization rely on the use of graphical modeling to capture the complex causal dependencies among variables of interest (represented as interconnected nodes in a graph structure). In the embodiments herein presented, hierarchical Bayesian networks are used to instantiate complete machine learning models, specifically a generative classifier and a generative prioritizer. The generative aspect of these machine learning models allows to explicitly model the structure of the causal process that generates the data, with a mixture of linear and non-linear dependencies, therefore providing a clear advantage for the classification of variants with a mixture of benign and pathogenic effects.

In addition to graphical modeling, both algorithms rely on the use of Bayesian statistics to estimate the probabilities associated with the causative process linked with generating the data. The embodiments herein presented are therefore significantly different from a pure statistical approach (e.g., logistic regression) which does not use explicit probability models.

### I. A Hierarchical Bayesian Network for Variant Classification

Variant classification is based on the observation that damaging or disease-causing genetic variants have distinctive biological features that differentiate them from tolerated or benign genetic variants. Variant classification involves examining features such as the genomic location of the variant and measures of the consequences of the variant (both direct and indirect) to determine whether the genetic variant is disease-causing or benign.

Existing Algorithms for Variant Classification and their Limitations

Previous approaches to variant classification have attempted to improve this process using different types of machine learning models (e.g., support vector machines such as CADD, random forest classifiers such as VEST, hidden Markov models such as FATHMM, deep neural networks such as MVP). However, the inherent nature of the problem of variant classification as well as the properties of the data used for variant classification limit the performance of existing machine learning approaches. First, the nature of a genetic variant being benign or pathogenic is influenced by several underlying biological processes. As a result, some genetic variants may be challenging to classify, as separate biological properties can lead to insufficient or conflicting evidence, which most machine learning algorithm are unable to quantify. Furthermore, variant classification algorithms are often trained on highly dimensional data sets with a large proportion of null or missing observations. This means that, as the number of dimensions (i.e., variables) increases, the data points available to train the model or to submit to statistical analysis quickly become insufficient (this problem is known as the “curse of dimensionality”). Finally, labelled data used to train variant classification algorithms are most often biased, which limits the performance of the algorithms when extrapolating beyond the training data. In summary, machine learning algorithms used for variant classification face the following limitations:
Poor performance in scoring or classifying variants with conflicting or insufficient evidence for pathogenicity;
Inability to quantify the amount of evidence supporting the prediction.
Poor performance in scoring or classifying variants with highly dimensional and sparse data;
Poor performance in extrapolating predictions to non-observed data sets due to bias in the training data.
   Advantages of Hierarchical Bayesian Networks in Combination with Biological Experimentation for Variant Classification

In contrast with existing methods, the machine learning approach disclosed herein involving hierarchical Bayesian networks in combination with biological experimentation provides a better approach to the task of variant classification due to the following:

### Ability of the Bayesian Framework to Quantify Evidence in Support of Null Hypothesis:

The Bayesian statistical framework provides the unique ability to quantify how much the data supports alternative hypotheses (e.g., whether the mutation is benign or pathogenic). This additionally enables the algorithm to deal with conflicting or inconsistent evidence during training and when making predictions. This is crucial in diagnostic reasoning since it allows computing posterior probabilities and confidence scores around the predictions, taking into account various aspects of the data (i.e., number of data points, causal explanatory value of individual observations). In other words, Bayesian statistics allows context and prior information to enter the model predictions, thus providing an appropriate probabilistic reasoning framework to distinguish predicted probability (e.g., 70% probability of being pathogenic and 30% probability of being benign) from the confidence of the prediction (e.g., 80% confidence that the prediction of 70% being pathogenic and 30% being benign is correct). The ability to obtain posterior probabilities and confidence scores which enable us to capture the importance of the data in making a certain prediction is critical when making predictions of variants of unknown significance in human genetics.

### Robustness of Bayesian Networks Against Highly Dimensional and Sparse Domains:

Hierarchical Bayesian networks are ideally suited to deal both with “the curse of dimensionality” and with sparse data in general. Bayesian networks are graphical models expressing the conditional dependence structure between variables. As a result, the algorithms used for training and inference need only to explore regions of the parameter space defined by the connectivity of the graph. This reduces the search space, which is vital when dealing with highly dimensional data as the search space grows exponentially with dimensionality. Second, Bayesian networks naturally accommodate missing observations and unobserved processes (i.e., variables for which we don't have direct observations but only indirect evidence), significantly alleviating the issues associated with data sparsity.

### The Connectivity of the Bayesian Network can be Determined Through a Human-Driven Generative Process to Overcome the Curse of Dimensionality:

Data-driven approaches often rely on algorithms or statistical procedures to ‘mine out’ the most probable structure of the graph (probability model) from the data and estimate parameters (probability distributions) associated with each variable (a node in the graph). Data-driven approaches have obvious benefits for automatized discovery tasks (i.e., finding the most likely model for a given domain) but are highly inefficient in domains where the curse of dimensionality is as prominent as variant classification and prioritization. The problem stems from the fact that the number of structures to evaluate grows exponentially with the number of variables (dimensions) in the data. This, in combination with high data sparsity, makes pure data-driven approaches to determine graph connectivity suboptimal.

Instead, the approach disclosed herein is human-driven; in a first step, human expert knowledge is used to generate a candidate graph structure reflecting current scientific knowledge of the genomic generative process (i.e., the underlying process generating the genomic data, understood as the genetic, biological and clinical properties of each individual variant). This provides a scientifically grounded plausible search space for estimating the graph structure with the highest explanatory power. Next, different variations on the candidate graph structure (e.g., deletion/additions of some links) are evaluated against the same dataset using standard metrics for model comparison (e.g., Bayesian information Criterion (BIC)). Three such candidate graph structures are illustrated in FIGS. 8A, 8B and 8C. Connections between nodes within the graph structure can be added and/or removed/pruned to improve the predictive performance of the model. For every model, the estimation of the model parameters (training) is carried out using standard learning algorithms (e.g., Expectation-Maximization (EM) algorithm). The model with the highest classification performance and the minimum amount of model complexity is then selected as the core graph structure to be used for classification. In some embodiments, additional training can be done to integrate novel information on top of previously trained information.

The Combination of Machine Learning with Biological Experimentation Addresses Issues with Data Sparsity and Bias in the Training Data:

A unique feature of an embodiment is that it uses a combination of machine learning methods and high throughput generation of molecular data through biological experiments. Biological experimentation allows for the generation of data reflecting the consequences of specific genetic variants. The biological experiments consist in generating, introducing and expressing a library of genetic variants in vitro to measure their molecular and/or cellular consequences. These measurements enable quantification of the molecular and/or cellular effects of genetic variants and can be used as input data to train the machine learning models for variant classification. As a result, one of the strengths of data generation through biological experimentation is the ability to overcome data sparsity by filling missing or null observations related to individual genetic variants. Furthermore, data generation through biological experimentation can provide additional information to directly support the classification of a large number of genetic variants as pathogenic or benign, thus overcoming existing bias in labelled data. The measurements of variant effects at the molecular and/or cellular level can be included in several ways as part of the input data used for training the Bayesian Network for variant classification in various embodiments. This is described in more detail further below.

A Cybernetic Loop Between the Hierarchical Bayesian Network and Biological Experimentation Enables Further Investigation into Low Confidence Predictions or Predictions with Insufficient or Conflicting Evidence:

The hierarchical Bayesian network generates predictions for novel (i.e., unobserved) variants and quantifies the support such evidence would provide for a given classification. In combination with biological experimentation, this allows the model to generate a list of potential molecular, cellular and/or clinical consequences for a specific genetic variant (and vice-versa), together with a probability reflecting the amount of evidence supporting this prediction. Genetic variants for which there is insufficient or conflicting evidence for the algorithm to make a prediction with high probability or high confidence can be selected from this list and further investigated experimentally (this is described in more detail further below). New biological data representing measurements of the molecular and/or cellular consequences of these genetic variants of interest can be generated experimentally and derived scores can be updated in the training data. This new information can be integrated in the parameters of the Bayesian network by performing an additional training step (i.e., incremental learning) using the previously trained network as a starting model. The result of the additional training with novel data is an increase in confidence of the predictions due to the increase in the evidence available to classify genetic variants.

The integration of novel information from data generated through biological experiments enables the hierarchical Bayesian network to capture relationships among different molecular properties of a genetic variant (i.e., finding generalizable patterns within the data, such as “when a genetic variant leads to this molecular alteration, this other feature takes a specific value, which had not been identified previously”). Thereby, the combination of biological data generation and hierarchical Bayesian network modelling for variant classification captures the molecular consequences of a genetic variant as a generative process. This feedback loop between biological experimentation and machine learning ultimately allows for a focus-driven approach to searching the space of possible causal relationships between a genetic variant and its molecular, cellular and/or clinical consequences.

Ia. Description of the Hierarchical Bayesian Network Used for Variant Classification

A hierarchical Bayesian network is used for variant classification. The Bayesian network model is defined by:
a. the set of variables/nodes (either observed or hidden), each with its own parameters (conditional probability distributions or CPDs)
b. the causal dependencies linking the nodes (variables) expressed as directed edges in the graph structure.

Being cast in a Bayesian statistical framework, the variant classification process inherits the advantages and benefits mentioned previously.

The input for process 100 is a patient's genetic data in the form of a VCF file listing the genetic variants identified in their DNA sample, together with their phenotype data in the form of a list of terms from an ontology describing clinical phenotypes (e.g., Human Phenotype Ontology (HPO)).

During both training of the network and use for classifying patient genomic data, an individual VCF file is read, formatted and annotated with genomic, biological and/or clinical features using a pipeline prior to entering the classification algorithm 110. First, this automated pipeline reads the list of genetic variants submitted in the VCF file and extracts information from each individual genetic variant for analysis. For instance, the pipeline can filter genetic variants from the list of variants to be analyzed, according to properties of individual genetic variants identified in the sample (e.g., whether the variant identified passes quality controls in sequencing and primary informatics analysis). Then, the automated pipeline annotates each individual genetic variant in the processed list with relevant genetic, biological and clinical information, using the genomic coordinates of the genetic variant as a reference. Examples of the genetic, biological and/or clinical features which are annotated for each genetic variant include the gene affected by the genetic variant, measures of evolutionary conservation or the reference amino acid on which the genetic variant is located (refer to Table 1 for further examples and Table 2 for a list of features annotated). Variant annotation is done by generating a table in which the first column contains the list of genetic variants to be processed, while each additional column corresponds to an individual genetic, biological and/or clinical feature.

FIG. 3 illustrates the format of input data for the classification algorithm 110 after formatting and annotation has been completed. A list of input features for the classification algorithm is further provided in Table 2. As can be seen, these data are sparse, so that, on many occasions, a full input data vector is not available. During use of the fully trained classification algorithm 100 the input data comprise a patient's genetic variant data, as determined by sequencing and initial informatics analysis of the sequencing data (i.e., after genetic variant calling). During normal use of the trained classification algorithm, the input genetic variant data will be annotated with the corresponding genetic, biological and/or clinical features using the pipeline mentioned above and discussed in more detail further below. Annotations include genetic, biological and/or clinical data that is relevant to classifying genomic variants, including MAVE experiment-derived data when available (discussed further below). Equally, during training of the classification algorithm, the input data will consist of genetic variant data annotated with the corresponding genetic, biological and clinical features, such as known classifications of (individual) variants, genetic, biological and/or clinical data that is relevant to classifying genomic variants and/or MAVE experiment-derived data when available.

The input data shown in FIG. 3 comprise a data matrix with a number of rows and columns. In the embodiment, each row relates to a genetic variant that is to be classified, with the columns providing details of the genetic variant itself. The column labels indicate the nature of the genomic feature quantified in the column in question.

FIG. 4 illustrates the process of automated variant classification (corresponding to step 110 in FIG. 2), consisting of variant annotation (corresponding to step 700 in FIG. 7), followed by variant classification (corresponding to step 710 in FIG. 7). FIG. 4 lists possible sources of input data for the annotation of genetic variants with genetic, biological and clinical features, including how the data generated by MAVE experiments feeds into these data.

The machine learning algorithm is a hierarchical Bayesian network comprising observed nodes (described in more detail below) that accept the various input data described before and hidden nodes that only accept input from other nodes of the network (both hidden and observed). A high-level example of the hierarchical Bayesian network 400 used in an embodiment, for classifying genetic variants is shown in FIG. 5. Input 410 is configured to receive input data. It will be appreciated that, during training of the network, these input data will be genetic variant data annotated with corresponding genetic, biological and clinical features, including experimental and computational data derived from MAVEs (when available), as described further below. It will equally be appreciated that, during the classification operation, input data will be genomic variant data, for example as provided in a VCF file, annotated with corresponding genetic, biological and clinical features, including experimental and computational data derived from MAVEs (when available). Annotated genomic variant data relating to a patient (an extract of an example is shown in FIG. 3) is applied to the network 400/input 410, one row/genetic variant at a time.

The observed nodes receiving the input data can be either nodes that have a connection with one or more child nodes, wherein the child node is causally dependent from the observed node, or from the observed node and one or more other observed or hidden nodes. Conversely, the observed node may itself be a child node of one or more other observed or hidden nodes, with a causal dependence between the observed node and its parent nodes. The network 400 is configured to derive the states of the hidden nodes based on these causal connections. In this manner, features that cannot be directly observed for an individual genetic variant and its corresponding annotations can be predicted in the trained network 400 and, based on the thus derived information, the likelihood of the genetic variant being pathogenic or benign can be derived.

The network 400 separates hidden nodes into those nodes 420 that relate to the constraints imposed by the genetic region of the genomic variant and those nodes 430 that relate to the consequences of a genetic mutation under consideration. Node 480 represents the functional disruption of the gene or gene product caused by the genetic variants being classified.

More specifically, the nodes 420 comprise one or more nodes 440 that relate to evolutionary conservation (i.e., regions of the genome, such as genes or segments of chromosomes, that are conserved amongst species and therefore may be functionally important) and one or more nodes 450 that relate to characteristics of genetic variation within the genomic region (i.e., regions of the genome, such as genes or segments of chromosomes where genetic variants are observed in humans).

The nodes 430 in turn comprise one or more nodes 460 that interpret the alterations at the RNA level and one or more nodes 470 that interpret the alteration at the protein level. These nodes 440 to 470 in turn inform one or more child nodes 480 that predict the functional disruption the genetic variant causes, for example alterations in RNA stability or splicing or a change in the stability or activity of a protein. These one or more hidden nodes 480 in turn have a causal connection with a node 490 that provides the desired characterization of the genetic variant into benign or pathogenic. Indications of the certainty of the model predictions (i.e., posterior probability of the predicted label and confidence score) are calculated per single prediction. Both the posterior probability of the prediction and the confidence score are expressed as percentages. The confidence score for single predictions is calculated from the posterior probability of the prediction and the credible interval. The confidence score for single predictions indicates how likely is the predicted classification as benign or pathogenic under the current data (i.e., how much does the available data support the characterization). For instance, a 80% likelihood of being benign could be accompanied by a 70% confidence in the prediction indicating further data is needed in order to make a very strong prediction with, for example, 95% confidence.

The connection between two nodes represents the statistical dependency between these two nodes, including the directionality of the causal influence. The statistical dependency between two nodes is captured by the flow of information from parent node to child node: first, evidence is provided to the parent node, which computes the probability of observing this evidence. Then, the evidence provided to the parent node flows to the child node, which computes the probability of each of the possible states in the child node, given the information provided by the parent node. If the child node has more than one parent node, the evidence from all parent nodes flows to the child node, to compute the probability of each of its possible states. If no evidence is directly input to a node, the node computes the probability of each of its possible states on the basis of available evidence from its parent nodes. The state with the highest probability is retained as the state of this node. Inference on the state of a child node can therefore be done on the basis of evidence in any of its parent nodes. Equally, inference on the state of any parent node can be done on the basis of evidence in any of its children nodes. Furthermore, inferences can be propagated throughout the system along any path within it (i.e., belief propagation).

Whilst nodes 440 to 470 and node 480 are illustrated in FIG. 4 as individual nodes, in one embodiment, one or more or each of these nodes are formed by a network of observed and/or hidden nodes representing or contributing to the above discussed function of the respective nodes. Any such network itself is, in one embodiment, a hierarchical Bayesian network. In one embodiment, the observed nodes within a network that forms the respective nodes 440 to 470 have as their input those input features shown in Table 2 that relate to the stated function of the respective node. It will be appreciated that, whilst in FIG. 4 a single connection is shown between the individual nodes 440 to 470 with node 480, in embodiments in which one or more of nodes 440 to 470 are represented by a network, each of the networks provides multiple inputs from individual nodes of the networks represented by nodes 440 to 470 to node 480.

Node 440, or the network represented by node 440, represents the evolutionary conservation of a genomic region in which a genetic variant being classified is found. The connection between node 440 and node 480 represents the statistical dependency between evolutionary conservation of a genomic region in which a genetic variant arises and the functional disruption to a genetic region, gene or gene product caused by this genetic variant. Node 440 receives input data pertaining to the evolutionary conservation of a genomic region in which a genetic variant being classified is found. Based on this, node 440 calculates the probability of observing this data and propagates the state with the highest probability of node 440 to node 480.

Node 450, or the network represented by node 450, represents the constraint to genetic variation in human population of a genomic region in which a genetic variant being classified is found. The connection between node 450 and node 480 represents the statistical dependency between constraint to genetic variation in human population of a genomic region in which a genetic variant arises and the functional disruption to a genetic region, gene or gene product caused by this genetic variant. Node 450 receives input data pertaining to the constraint to genetic variation in human population of a genomic region in which a genetic variant being classified is found. Based on this, node 450 calculates the probability of observing this data and propagates the state with the highest probability of node 450 to node 480.

Node 460, or the network represented by node 460, represents the effects at the RNA level of a genetic variant being classified. The connection between node 460 and node 480 represents the statistical dependency between the effects at the RNA level of a genetic variant and the functional disruption to a genetic region, gene or gene product caused by this genetic variant. Node 450 receives input data pertaining to the effects at the RNA level of a genetic variant being classified. Based on this, node 460 calculates the probability of observing this data and propagates the state with the highest probability of node 460 value to node 480.

Node 470, or the network represented by node 470, represents the effects at the protein level of a genetic variant being classified. The connection between node 470 and node 480 represents the statistical dependency between the effects at the protein level of a genetic variant and the functional disruption to a gene or gene product caused by this genetic variant. Node 470 receives input data pertaining to the effects at the protein level of a genetic variant being classified. Based on this, node 470 calculates the probability of observing this data and propagates the state with the highest probability of node 470 to node 480. As mentioned above, node 480 represents the functional disruption of the gene or gene product caused by the genetic variants being classified. Node 490 represents the classification of a genetic variants as pathogenic or benign, (alternatively, as damaging or tolerated). The connection between node 480 and node 490 represents the statistical dependency between the functional disruption of a genetic region, gene or gene product caused by a genetic variant and its classification as pathogenic or benign. Node 480 receives from nodes 440, 450, 460 and 470 the probabilities calculated by each node. Based on these values, node 480 calculates the probability of a genetic variant leading to functional disruption to a genetic region, gene or gene product and propagates the state with the highest probability of node 480 this value to node 490. Node 490 receives the state with the highest probability as calculated by node 480. Based on this value, node 490 calculates the posterior probability of a genetic variant being pathogenic or benign.

Observed nodes as well as hidden nodes may have various discrete states that can be adopted by the node or, for suitable biological properties (e.g., evolutionary conservation scores or distance in base pairs to the closest splice site), may be able to adopt any particular value within a continuous range. In the trained network, the probabilities associated with the states of a variable reflect the knowledge extracted from training in the form of a-priori expectations (probabilities derived from the training data during training of the model and before providing input data). FIG. 6 illustrates an example of a node having such a-priori expectations over the states of that particular variable. Whilst the node shown in FIG. 6 relates to an observed node (namely which type of splice site is affected by the observed genetic variant) it will be appreciated that hidden nodes may equally adopt discrete states. Moreover, it will be appreciated that the number of discrete states is only dependent on the variable interpreted by the node. The node shown in FIG. 6 is connected to one other node (not shown).

The predictions of the states of individual nodes in the network 400 are estimated by applying Bayes Rule to calculate the posterior predicted probabilities for any given node given the observed data, the model connectivity and the estimated parameters.

An example of the classification process is illustrated in FIG. 7. As is shown in this Figure, the classification algorithm can have two different phases, a first phase 610 in which it is trained and a second phase 620 in which the trained classifier is used to classify genetic variants.

The training phase 610 itself has two phases, an initial phase 630 in which an initially untrained machine learning model is trained to enable it to classify correctly and an ongoing training phase 640 in which an already functional (and possibly also operational) model is continuously re-trained and the original training refined using newly available training data. In an embodiment, in both training phases the same steps are performed. The training is based on initial or new raw training data that is received in step 650. These data are then annotated in step 660 (discussed in more detail below) and pre-processed in step 670 (also discussed in more detail below) before they are used in training the machine learning classifier in step 680.

The training data sets include subsets of an annotated list of all possible single nucleotide variants (SNVs) in the human genome, of all possible single nucleotide insertions and deletions in the human genome, of all multi-nucleotide and all small insertion/deletion variants (<100 base pairs or bp) observed in humans. For each genetic variant, annotations with additional genetic, biological and/or clinical information on the mutation (e.g., gene or genomic region, evolutionary conservation, frequency of mutation observed in the population . . . ) are also provided. When available, data generated by high-throughput biological experiments (such as MAVEs) is used to annotate individual genetic variants with experimentally determined measurements of molecular and/or functional consequences. Additionally, this experimentally generated data can be analysed computationally to derive molecular and/or functional scores, which can be then be used to annotate individual genetic variants. Molecular and functional scores are computationally determined scores reflecting the severity of the effect of a genetic variant on a specific molecular property of a gene or gene product, such as a measure of the effect of the genetic variant on RNA levels, RNA splicing, protein stability and protein functions, which can be generated by a machine learning algorithm trained using data generated by MAVEs as input data. Each training (or indeed later each operational classification) step operates on a single genetic variant. An example of a small number of rows of input data, after annotation and pre-processing in steps 660 and 670 shown in FIG. 7, is shown in FIG. 3, as discussed above. As can be seen from this Figure, the data is very sparse so that situations in which no input data is available for many observable variables are routine. Both observable and hidden nodes can have a default probability distribution associated with the state they can adopt. The splice_type node shown in FIG. 6, for example, defaults to a high probability that the genetic variant in question does not affect a splice site (i.e., “none”) in the absence of input information to the contrary. This assumption is made based on a probability distribution (as shown in FIG. 6) the node has acquired during training. Concrete input information for an observable node overrides this assumption.

The annotation pipeline is then used to annotate unlabeled data 690. The data includes some or all genetic variants of a patient as determined by sequencing of genomic material and initial informatics analysis. In an embodiment, these data are in the form of a standard VCF file, as is in already use in the field. The data is annotated with additional genetic, biological and/or clinical information available on the mutation (e.g., gene or genomic region, evolutionary conservation, frequency of mutation observed in the population, experimentally determined molecular and functional consequences of the mutation as obtained by MAVEs (when available) or computationally derived molecular and/or functional scores). The trained model, based on the annotated data 700, characterizes the variants in step 710 and outputs a list of characterized genetic variants in step 720. The characterized list may be presented in the format of a standard VCF file that comprises the relevant annotations, the classification as benign or pathogenic, the associated probability of the prediction and/or the confidence score of the prediction.

With regard to the individual steps shown in FIG. 7, the raw data received in step 650 consists of a list of genetic variants, which are subject to annotation (step 660). The annotation step 660 performed during training is the same as the annotation step 700 performed during production use of the trained model. After annotation, the annotated data may be sparse and may have the format shown in FIG. 3. In step 670 the input training data is prepared for the model analysis by sampling subsets of annotated genetic variants from the list of all genetic variants annotated. Genetic variants can be sampled at random from the list of all variants to generate training data sets with random features. Alternatively, genetic variants can be sampled from the list of all variants according to specific genetic, biological and/or clinical features (e.g., type of genetic variant, genomic location, evolutionary conservation) to generate training data sets with specific genetic, biological and/or clinical features.

In embodiments the following data features are used to make graded predictions on the pathogenicity of a genetic variant:

1. Constraint or tolerance to variation of the genetic region (node 420)
Evolutionary conservation (node 440) deriving inputs from, for example:
   Evolutionary conservation scores (observed nodes)
   Presence of domains or motifs (observed nodes)
Genetic variation observed in human population (node 450) deriving inputs from, for example:
   Mutation density in surrounding regions (e.g., windows of 100 base pairs, 1000 base pairs, 10,000 base pairs) (observed nodes)
   Distance to the closest mutation in base pairs (observed nodes)

2. Effects of genetic variant (node 430)
Alterations at the RNA level (node 460) deriving inputs from, for example:
   Predicted consequence of the variant (e.g., nonsense mutation, frameshift mutation, splice site mutation) (observed nodes)
   Prediction of nonsense-mediated decay (observed nodes)
   Scores for prediction of splicing alterations (observed nodes)
   Direct measurements of RNA properties generated experimentally through MAVEs (e.g., RNA levels, splice site selection) (observed nodes)
   Molecular and/or functional scores of RNA properties generated computationally through machine learning, trained using experimental data such as MAVEs (e.g., computational prediction scores of RNA levels, splice site selection) (observed nodes).
Alterations at the protein level (node 470) deriving inputs from, for example:
   Predicted consequence of the variant (e.g., missense variant, inframe deletion, stop lost) (observed nodes)
   Scores and measurements of the predicted tolerance to amino acid change in the case of missense variants (e.g., Grantham score, BLOSUM62, comparison of biochemical properties) (observed nodes)
   Measurements of protein properties generated experimentally through MAVEs (e.g., protein levels, protein stability) (observed nodes)
   Molecular and/or functional scores of protein properties generated computationally through machine learning, trained using experimental data such as MAVEs (e.g., computational prediction scores of protein levels, protein stability) (observed nodes).

The information from node 420 and 430 is integrated in the hidden node 480, which predicts whether the variant observed is damaging to its corresponding genetic region, gene or gene product (known as functional disruption). Based on the information from node 480, node 490 then classifies the genetic variant as benign or pathogenic. Of the abovementioned data features used, direct measurements of molecular effects of genetic variants on a genetic region, gene or gene products (e.g., RNA properties, protein properties and protein function) are considered strong evidence for predicting the pathogenicity of a genetic variant, compared to scores derived from computational predictions.

The frequency of a genetic variant in individuals affected by a specific genetic disorder versus healthy individuals is the most informative parameter as it allows to directly classify a mutation as pathogenic or benign for a condition. However, since most genetic variants are either rare or have never been observed, this information is missing for most genetic mutations (>99.99% of all possible mutations in the human genome).

To address the problem of missing data, the approach adopted in embodiments relies on generating data to measure the molecular effects of thousands of mutations at a time using high-throughput biological experiments and then leveraging on the hierarchical Bayesian network approach to extract the relevant information and inform inferences on novel observed or unobserved mutations. The process used to generate these data is described in more detail below.

### Definition of Graph Structure (Connectivity) Via Expert Human Knowledge

After defining the variables to be included, the next step in generating the Bayesian network is the specification of the connectivity graph linking variables (observed and hidden) together. Existing data-driven algorithms for automatic detection (i.e., graph structure mining) are extremely limited in this case given the vast number of possible graphs with different structures (due to the curse of dimensionality, the number of possible graphs with different structures scales exponentially with number of features or nodes to be included).

The approach used in embodiments relies on translating human-level expertise into specific connectivity profiles in the hierarchical Bayesian network. This consists in translating the genetic, biological and/or clinical features directly causative for the pathogenicity of a genetic variant into variables, represented as nodes in the hierarchical Bayesian network. Then, the relationship between these features is translated into connections between the nodes, representing the causal influence between the features. For this, first, a basic graph structure reflecting the causal influences among variables (features) is defined as the candidate graph structure of the genomic generative process. This candidate structure is created by translating human-expert knowledge of the genomic process into a connectivity graph which reflects causal dependencies between variables. Second, different variations of this candidate structure are created by modifying some parts of the graph (i.e., sub-graphs) of the candidate structure using human-expert knowledge. Three examples of different variations of the candidate structure are presented in FIGS. 8A, 8B and 8C, in which parts of the graph are modified using human expert knowledge. Graph structures may differ in the type of nodes they include and/or in the manner in which the nodes are connected. The manner in which the nodes are connected may differ between the various candidate graphs in one or more of:

the direction of information flow between nodes; and

direct or indirect (i.e., via one or more other nodes) connection between nodes.

Variations in the structure of the model are then compared to the original structure in terms of the explanatory power of the model through cross-validation. First, each model is trained on the same data set until convergence, after which the overall classification accuracy of the model is evaluated on a test data set and the mutual information (MI) score between nodes is calculated. The MI score is a measure of the causal influence exerted by one variable over another (i.e., how well does the information present in one variable explain the information another variable bears). This procedure is conducted in order to remove unnecessary or erroneous causal dependencies marked by an extremely low MI score (i.e., connections with very low predicted causal influence). The MI score between two random variables (X,Y) is calculated with the following equation:

MI(X;Y)=D_{KL}(P_{(X,Y)}∥P_{X}⊗P_{Y})

with the overlap between any two variables (X, Y) indicated by MI(X;Y) being given by the Kullback-Leibler divergence (D_{KL}) of the intersection P_{(X,Y)} of the marginal probabilities of the two variables, P_{X} and P_{Y} respectively. When the two variables are independent (no mutual information) the joint probability distribution, P_{(X,Y)}, is equal to 0, increasing the more mutual information is present.

In one embodiment, connections with MI scores <0.0001 are assumed to be null and removed. In a further step, this process of creating variations on the candidate structure by maximization of the MI scores (after training) for all pair of nodes is conducted multiple times until the resulting model exhibits a satisfactory overall classification accuracy (e.g., >95%). In one embodiment, local connectivity of specific regions of the graphs where the MI score is significantly low is adjusted to provide a further candidate graph for training and testing in the manner described above. In an embodiment, this process is performed multiple times until the hierarchical Bayesian network reaches a satisfactory overall performance, for example as expressed by a predetermined desired overall classification accuracy. In one embodiment, this accuracy is >97%.

After having specified the graph structure, the conditional model parameters (i.e., the parameters of the conditional probability distributions or CPDs) for each node are estimated from the training dataset. In this step, training data (i.e., lists of genetic variants throughout the human genome annotated with different genetic, biological and clinical features, including experimental measurements of molecular effects derived from MAVEs (where available) and molecular and/or functional scores derived computationally from MAVE data, and labelled as pathogenic or benign where available) are entered in the model, allowing for the model to derive structured predictions across the data. Different algorithms can be used to estimate the CPDs such as Junction-tree algorithm, Expectation-Maximization (EM) algorithm or Markov-Chain Monte-Carlo sampling. In one embodiment, the trained model is validated with a cross-fold validation procedure (k=2,3,5). This process is repeated multiple times to yield an overall statistical measure of the goodness-of-fit of the model under different partitioning schemes of the training data.

After validation, the trained model is ready to be used to generate predictions for new (unseen) data and classify genetic variants as pathogenic or benign.

### Label Inference, Prediction Probability and Confidence Scores Via Bayesian Algorithms

The last step of the procedure is to submit new observations (i.e., patient data) to the trained model and compute the predicted classification probabilities along with confidence measures. For each variant, the inference algorithm returns the posterior probabilities for each of the possible labels (e.g., benign/pathogenic), which represent the predicted classifications. The predicted posterior probabilities express how likely a particular label is under the current data and thus provide not only the predicted label with highest likelihood for that particular data vector (i.e., the classification) but also provide the posterior probability of the prediction, indicating how much the evidence provided supports that particular classification. In addition to the posterior probability, a confidence score can be derived for each individual prediction, calculated from the posterior probability and the credible interval. A second confidence score can also be computed globally to quantify the overall precision of the model. This global confidence score is computed as the ratio between the likelihood of observing the data under the hierarchical Bayesian network model over the likelihood of observing the same data under a baseline model (in this case, a random linear classifier). For all of the model comparisons described herein, the Bayesian information criterion (BIC) measure was used to quantify how well a model predicts the data and thus to compare different models (model comparison).

### Input Data for the Bayesian Classification Network

### Training Data

A variant database containing a list of all possible single nucleotide variants (SNVs) in the human genome, of all possible single nucleotide insertions and deletions in the human genome and of all multi-nucleotide, insertion and deletion variants (<100 bp) previously observed in humans is used to generate training data sets for the hierarchical Bayesian network for variant classification. Each variant in the database is annotated with additional genetic, biological and clinical information available, including computational predictions, measurements from biological experiments (i.e., MAVEs) and observations from human data. Subsequently, subsets of annotated variants within the database are selected randomly to generate training data sets for initial or incremental training of the hierarchical Bayesian network. Subsets of annotated variants within the database may also be selected according to specific genetic, biological and/or clinical features to generate training data sets for initial or incremental training of the hierarchical Bayesian network.

A bespoke bioinformatics pipeline for digital saturation mutagenesis of a genomic region of interest is initially used to generate the list of genetic variants to include in the training data for the hierarchical Bayesian network for variant classification (i.e., the raw training data 650 in FIG. 7). FIG. 9 shows a flowchart describing the processing steps in this pipeline 900. In a first step 910 this pipeline takes as input a FASTA file, which contains the coordinates and DNA sequence of a genomic region of interest (e.g., a specific gene or list of genes, a specific locus, genomic region or list of genomic regions, a chromosome or a genome). In step 920 the bioinformatics pipeline 900 then processes this information to generate a list of all possible single nucleotide variants, insertions and deletions in the genomic region of interest, following the VCF file format (chromosome, reference position, reference base, alternate or mutant base in a tab-delimited format). The small multi-nucleotide variants, insertions and deletions (<100 bp) previously observed in humans are collected and curated from publicly available databases of genetic variation in humans (e.g., 1000 genomes project, gnomAD).

In a further step 930, the list of variants in VCF file format are used as input for a second bioinformatics pipeline 940, which annotates each variant with relevant biological information (step 660 in FIG. 7, where the features are annotated per specific mutation, genomic position, gene, genomic region or amino acid, depending on the specific annotation that is added to the variant). First, the variant annotation pipeline uses a pipeline for functional annotation of genetic variants such as the Variant Effect Predictor tool from ENSEMBL (VEP: https://www.ensembl.org/info/docs/tools/vep/index.html) or the ANNOVAR software tool (cited, for example in https://www.nature.com/articles/nprot.2015.105) to annotate the list of variants of interest using the default annotations. Then, the variant annotation pipeline retrieves information pertaining to each variant from different data sources including data from biological experiments (i.e., MAVEs) and computationally-derived molecular and/or functional scores, as listed in Table 1 and Table 2 (see below). The output of the annotation pipeline 940 is a list of variants in the genomic region of interest annotated with relevant biological information (step 950), including data pertaining to evolutionary conservation, variation in human population and RNA and protein features (both from biological experiments and computationally derived scores).

The data undergoes further processing in step 960 (also shown as step 670 of FIG. 7), prior to being input in the model, in which a subset of genetic variants is sampled from the list of all annotated genetic variants. Genetic variants can be sampled at random from the list of all variants to generate training data sets with random features. Alternatively, genetic variants can be sampled from the list of all variants according to specific genetic, biological and/or clinical features (e.g., type of genetic variant, genomic location, evolutionary conservation) to generate training data sets with specific genetic, biological and/or clinical features.

The output of this process provided in step 970 is a list of variants in VCF format with genetic, biological and clinical annotations, as shown in FIG. 3, which can be input as training data in the classification algorithm.

### Input Data

During operational use of the classification network, the input data is the list of genetic variants identified in the patient sample in a VCF file format as shown in step 1010 of FIG. 10. In step 1020 this list of genetic variants is annotated with relevant genetic, biological and/or clinical features as listed in Table 1 and Table 2 (see below) (step 700 in FIG. 7, where the features are annotated per specific mutation, genomic position, gene, genomic region or amino acid, depending on the specific annotation that is added to the variant) in the same way as described above for the training data with reference to step 940 of FIG. 9. The output of this process provided in step 1030 is a list of variants in VCF format with genetic, biological and clinical annotations, as shown in FIG. 3, which can be input for analysis in the classification algorithm. In step 1040 this list is provided as input data to the classification algorithm described herein.

### Data Sources Used for Variant Annotation

The genetic, biological and clinical features used as annotations include predictions of the variant consequences at DNA, RNA and protein level, variant frequency in different populations, evolutionary conservation and clinical interpretation of the variant.

TABLE 1
Examples of annotations included in the variant database

| Genomic location | Genomic annotations from VEP |
|---|---|
| Mutation type | Mutation consequence annotations from VEP |
| Evolutionary | Evolutionary scores, protein domains, DNA motifs. |
| conservation | |
| Genetic variation | Variant allele frequency, variant density, |
| in human population | intolerance scores, regional constraint scores. |
| RNA features | Nonsense-mediated decay, distance to the closest |
| | splice site, type of splice site, splice prediction |
| | scores |
| Protein features | Amino acid chemical properties, Grantham score, |
| | BLOSUM62. |
| Clinical | ClinVar and gene-specific databases |
| interpretation | |
| Experimental | Experimental measurements of molecular |
| measurements | consequences obtained from biological experiments |
| | (i.e., MAVEs) |
| Molecular and/or | Molecular and/or functional scores computationally |
| functional scores | derived from data generated by MAVEs. |

The datasets used to generate these annotations are obtained from public servers in the case of publicly available data, from private servers in the case of licensed data or generated in case of proprietary experimental or computational data. The datasets are formatted and the relevant information is either directly extracted (e.g., PhyloP and phastCons scores per genomic position for evolutionary conservation) or otherwise derived from these data sets (e.g., regional mutation density is derived from allele frequency for mutations within a genomic region). Afterwards, the data is formatted (e.g., binning, scaling, discretization) in preparation for model analysis.

TABLE 2
List of annotations used as input features for the classification algorithm

| Data | Use | Information provided |
|---|---|---|
| most_sev_conseq | Mutation ID | Most severe consequence of the mutation (priority selected |
| | | by potential impact) |
| splice_type | Splicing | Closest splice site is ACCEPTOR or DONOR |
| Dst2Splice | Splicing | Distance to splice site in 20 bp; positive: exonic, negative: |
| | | intronic |
| Grantham | Protein | Grantham score |
| | biochemistry | |
| BLOSUM62 | Protein | Frequency of amino acid interchangeability |
| | biochemistry | |
| MW | Protein | Molecular weight of amino acid |
| | biochemistry | |
| VdW | Protein | Van der Waals surface of the amino acid |
| | biochemistry | |
| hydrophobicity | Protein | Amino acid hydrophobicity |
| | biochemistry | |
| hydroscore | Protein | Amino acid hydrophobicity score |
| | biochemistry | |
| p_isolectric | Protein | Amino acid isoelectric point |
| | biochemistry | |
| Side chain bonds | Protein | Number of hydrogen bonds in the amino acid side chain |
| | biochemistry | |
| Flexibility | Protein | Flexibility of the amino acid side chain |
| | biochemistry | |
| flex_score | Protein | Flexibility score of the amino acid side chain |
| | biochemistry | |
| charge | Protein | Electric charge of the amino acid |
| | biochemistry | |
| Biochemical | Protein | Special biochemical properties of the amino acid |
| | biochemistry | |
| TFBS | DNA motif | Number of overlapping transcription factor binding sites |
| pDom | Protein | Protein domains |
| | domains | |
| pModif | Protein | Post-translational protein modifications |
| | modification | |
| pDisulf | Protein | Protein disulfide links |
| | disulfide bonds | |
| pOther | Protein other | Other protein regions of importance |
| mamPhastCons | Evolutionary | Mammalian PhastCons conservation score |
| | conservation | |
| mamPhyloP | Evolutionary | Mammalian PhyloP score |
| | conservation | |
| priPhastCons | Evolutionary | Primate PhastCons conservation score |
| | conservation | |
| priPhyloP | Evolutionary | Primate PhyloP score |
| | conservation | |
| verPhastCons | Evolutionary | Vertebrate PhastCons conservation score |
| | conservation | |
| verPhyloP | Evolutionary | Vertebrate PhyloP score |
| | conservation | |
| ExAC_pLI | Gene | Intolerance to gene haploinsufficiency |
| | constraint | |
| ExAC_pRec | Gene | Intolerance to biallelic gene loss of function |
| | constraint | |
| ExAC_pNull | Gene | Tolerance to biallelic gene loss of function |
| | constraint | |
| Dist2Mutation | Population | Distance between the closest gnomAD SNV up and |
| | data | downstream |
| Freq10000 bp | Population | Number of frequent SNVs (AF > 0.05) found in gnomAD in |
| | data | a surrounding window of 10000 bp |
| Freq1000 bp | Population | Number of frequent SNVs (AF > 0.05) found in gnomAD in |
| | data | a surrounding window of 1000 bp |
| Freq100 bp | Population | Number of frequent SNVs (AF > 0.05) found in gnomAD in |
| | data | a surrounding window of 100 bp |
| Rare10000 bp | Population | Number of rare SNVs (AF < 0.05) found in gnomAD in a |
| | data | surrounding window of 10,000 bp |
| Rare1000 bp | Population | Number of rare SNVs (AF < 0.05) found in gnomAD in a |
| | data | surrounding window of 1000 bp |
| Rare100 bp | Population | Number of rare SNVs (AF < 0.05) found in gnomAD in a |
| | data | surrounding window of 100 bp |
| Sngl10000 bp | Population | Number of single-occurrence SNVs found in gnomAD in a |
| | data | surrounding window of 10,000 bp |
| Sngl1000 bp | Population | Number of single-occurrence SNVs found in gnomAD in a |
| | data | surrounding window of 1000 bp |
| Sngl100 bp | Population | Number of single-occurrence SNVs found in gnomAD in a |
| | data | surrounding window of 100 bp |
| ClinVar | Human | Clinical classification of the genetic variants |
| | disease | |
| MAVE_prot | Functional | Data from MAVE experiments measuring molecular effects |
| | protein data | of genetic variants on a protein. For example, the BRCA1 |
| | | data from Findlay et al 2018 (as below). |
| MAVE_RNA | Functional | Data from MAVE experiments measuring molecular effects |
| | RNA data | of genetic variants on RNA. For example, the BRCA1 data |
| | | from Findlay et al 2018 (as below). |
| MAVE_prot_imp | Molecular | Molecular and/or functional scores of effects on protein, |
| | and/or | imputed through computational analysis of MAVE data sets. |
| | functional | |
| | scores | |
| MAVE_RNA_imp | Molecular | Molecular and/or functional scores of effects on RNA, |
| | scores | generated through computational analysis of MAVE data |
| | | sets. |

The annotation listed in Table 2 are input features for the hierarchical Bayesian network used for classifying genomic variants. These features accordingly correspond to observed nodes of the hierarchical Bayesian network and connect to hidden nodes of this network. It will be appreciated that it is not essential for all of the input features listed in Table 2 to be used in the hierarchical Bayesian network. A hierarchical Bayesian network may equally be expanded to incorporate further input features/nodes as deemed expedient with improving knowledge in the field.

### II. Multiplexed Assays for Variant Effects (MAVEs)

As described above, the hierarchical Bayesian network for variant classification is trained with data, including biological data reflecting the observed and predicted functional consequences of thousands of genetic variants. This includes genetic variants in coding regions of the genome, such as missense and splicing mutations and genetic variants in noncoding regions of the genome such as promoters and regulatory elements. These biological data are generated experimentally using high-throughput biological methods (i.e., Multiplex Assays of Variant Effects or MAVEs) designed to measure the effect at the molecular and/or cellular level of hundreds or thousands of mutations in parallel. The measurements of variant effects at the molecular and/or cellular level can be included in several ways as part of the input data used for training the hierarchical Bayesian network for variant classification, as discussed above.

High-throughput biological methods to generate biological data can be used to create a catalogue of mutations with a functional interpretation prior to their identification in a patient sample. In one embodiment, the molecular and/or cellular measurements of variant effects can be used directly to support the novel classification of genetic variants according to guidelines from the American College of Medical Genetics, as part of the criteria for functional evidence demonstrating that a variant leads to abnormal or normal gene and/or protein function (PS3/BS3 criterion). This allows for new genetic variants to be classified as benign and/or damaging, thus increasing the number and unbiasing the data sets of classified (i.e., labelled) genetic variants. In this way, MAVEs can be used to create a catalogue of mutations with a functional interpretation prior to their identification in a patient sample.

In one embodiment, the biological data used to train the classification machine learning model is derived from wet lab experiments quantifying the effect of thousands of genetic mutations on different biological processes within a cell (including RNA transcription, RNA splicing, RNA stability, RNA degradation, protein translation, protein stability, protein function and protein stability). In one embodiment, the molecular and/or cellular measurements of variant effects are processed and normalized to be used directly as features within the training data set, serving as input for one of the nodes of the hierarchical Bayesian Network for variant classification (i.e., as for instance, other features included such as measures of evolutionary conservation).

In one embodiment, the molecular and/or cellular effects of genetic variants can be processed and used to train an intermediate specialized machine learning model, from which predictions for scores are generated. These predicted scores are used, in turn, as a feature in the input data to train the Bayesian Network for variant classification. For example, if the biological experimentation carried out consists in measuring the effects of genetic variants on mRNA levels, these measurements can be processed and used as part of the input training data for a machine learning algorithm specialized in predicting the effects of genetic variants on mRNA levels. Here, the measurements obtained from biological experiments can be used as a predicted label or score for the effect of genetic variants on mRNA splicing. After training, the specialized machine learning algorithm can then predict the effect of unobserved genetic variants on mRNA levels as a label or score. These predicted scores or labels for mRNA levels can then be used as input data for the Bayesian Network for variant classification, representing a feature to be used as input in one of the nodes of the network. This approach aims to provide insights into the biological rules underlying molecular and/or cellular processes (such as in this example, which mRNA sequences are detected by the splicing machinery of the cell as indicating the beginning of an intron to be spliced out of an mRNA transcript).

FIG. 11A is a scheme describing the general experimental outline of a MAVE, while FIG. 11B provides an illustration for each step of the process. Different methods can be used to generate, introduce, assay and quantify the effects of the variants of interest in the studied cells. The general outline of a MAVE experiment is as follows:
Variant library generation:

Generation of a genetic variant or gRNA library (1) and introduction into a population of eukaryotic cells (2). The product of these two steps is a population of genetically modified eukaryotic cells where each cell harbors one of hundreds or thousands of different variants in one or more genes or genomic regions of interest.
Stratification of variant effects:

Multiplexed functional assay (3) and quantification of variant effects (4). In this step, the genetically modified cells from step 2 may be subjected to functional assays. This leads to the selection of cells according to the consequences of the genetic variants they harbor, which are then quantified through DNA or RNA sequencing of the cell population. Alternatively, the genetically modified cells from step 2 may be propagated through cell culture, processed and harvested for molecular and/or cellular analysis, through for example, DNA or RNA sequencing of single cells or of the cell population.

In the following section, we provide additional information on each of these steps.

### Variant Library Generation

FIGS. 11A and 11B, Step 1; Construction of Variant Library:

This first step consists in generating a variant library, which is a library of synthetic DNA molecules in which all possible nucleotide substitutions of interest for a selected gene, genomic region or genomic locus are represented, with each DNA molecule carrying one or more variants. Depending on the method through which the variant library is introduced into a population of cells, there may be additional steps required to amplify and/or clone the variant library into a suitable plasmid for transfection or viral transduction. Additionally, the construction of the variant library may include a step in which a molecular barcode is introduced and linked to a genetic variant, to facilitate sequencing and analysis steps.

The variants included in the library can be single nucleotide substitutions as shown in FIG. 12, multiple nucleotide substitutions, nucleotide insertions, nucleotide deletions or random sequences of different lengths. Variant libraries can consist of hundreds up to hundreds of thousands of DNA molecules with different sequences.

The aim of this step is to generate a high-quality variant library in an efficient and cost-effective manner.

In one embodiment, the DNA variant libraries are designed in-house and ordered as oligonucleotides from companies such as Twist Bioscience (https://twistbioscience.com/) or IDT. The benefits of this method include:
High control over composition and uniformity of the variant library.
Low error rate in the synthesis of the variant library.
Highly scalable and cost-effective method.

An overview and comparison of different approaches to generate variant libraries is presented in Table 3.

TABLE 3
Comparison of different methods for variant library construction.

| | Control over | | | |
|---|---|---|---|---|
| | composition | | | |
| | of the variant | Error | Scal- | |
| Method | library | rate | ability* | Cost |
| Oligonucleotide synthesis | +++ | −/+ | +++ | +/+++ |
| Doped oligonucleotides | + | + | +++ | ++ |
| (oligos with degenerate | | | | |
| sequences) | | | | |
| Array-based DNA synthesis | ++ | + | ++ | ++ |
| (e.g., Custom Array) | | | | |
| Mutagenesis by Integrated | + | + | − | + |
| TiIEs (MITEs) | | | | |
| Random mutagenesis by error- | − | ++ | − | − |
| prone PCR of a template | | | | |

*Scalability is defined as a measure of the necessary hands-on work for each approach. Depending on the method through which the variant library is introduced into a population of cells in step 2 of FIGS. 11A and 11B, there may be additional steps required to amplify and/or clone the variant library into a suitable construct.

Alternatively, a library of gRNAs for a CRISPR-Cas system can be used as a variant library for multiplex targeting of several regions of interest throughout the genome, with each DNA molecule representing a different gRNA. Depending on the method through which the CRISPR-Cas system is introduced into a population of cells, there may be additional steps required to amplify and/or clone the gRNA library into a suitable plasmid for transfection or viral transduction, either alone or in combination with the CRISPR.

FIGS. 11A and 11B, Step 2; Delivery of Variant Library:

The aim of this step is to introduce variants from the variant library into a population of cells, by transfection (via episomes or electroporation) or lentiviral transduction of one or more DNA constructs into the cell. Delivery of a variant library to a population of cells can lead to temporary presence of individual variants of the library in the cell or to permanent presence of individual variants of the library in the cell, through integration, recombination or otherwise editing of the endogenous DNA sequence of the cell. In some embodiments, the cells to which the variant library is delivered may have been previously genetically modified or chemically treated to increase the likelihood of individual variants becoming permanently integrated in the cell's genomic sequence. It is desirable to have a genetically heterogeneous cell population where each cell carries or expresses a different variant, although whether or not this is achieved depends on the type of genomic region being evaluated and the method of delivery.

MAVEs can be carried out in different types of eukaryotic cells, including yeast, mammalian or human cell lines.

In one embodiment, this step consists of editing the genome of cells, to introduce and study the genetic variants or genetic alterations of interest in their native genomic context. To achieve this, cell lines are transfected with CRISPR/Cas9 constructs targeting the locus of interest, together with the variant library in the form of pooled DNA plasmids or single stranded oligonucleotides (ssODN). CRISPR/Cas9 induces a double strand break at the locus of interest, leading to homology-directed repair using the ssODN or DNA plasmids a template, as shown in FIG. 13. In some cases, the cell to which the DNA construct is delivered, may be chemically treated or genetically modified to increase the probability that the variant or variants carried by construct is integrated in the cell's endogenous DNA. The result is the integration of the variant in the endogenous locus of the cell (Findlay et al. Saturation editing of genomic regions by multiplex homology-directed repair. Nature 513, 120-123 (2014); Findlay et al. Accurate classification of BRCA1 variants with saturation genome editing. Nature 562, 217-222 (2018), the entireties of which are incorporated herein by reference). The benefits of this approach are as follows:
Higher quality of the data generated: studying variants in their native genomic context offers a better representation of their biological effects and bypasses some technical limitations associated with overexpression of a construct. For instance, it enables the possibility to study:
   Effects of variants in different mRNA transcript and protein isoforms.
   Effects of variants on mRNA translation, stability and splicing.
   Effects of mutations in non-coding or untranslated regions, for which the native genomic context is crucial.
The mutation of interest is permanently integrated in the genome, which allows for longer functional assays and therefore increases the flexibility in assay choice.

In another embodiment, another strategy to introduce the variants of interest in a cell is to deliver a copy of the gene or genomic region carrying the variant of interest (e.g., overexpression of a mutant construct transfected in a plasmid or transduced in a lentivirus). In some cases, the cell to which the DNA construct is delivered, may be chemically treated or genetically modified to increase the probability that the variant or variants carried by construct or the construct itself is integrated in the cell's endogenous DNA. A comparison of these approaches to introduce variants from variant libraries into cells is shown in Table 4.

TABLE 4
Comparison of different methods to introduce
a variant library into cells.

| | Quality of the | Flexi- | Costs and |
|---|---|---|---|
| | data and mapping | bility | required |
| | to biological | of assay | infra- |
| Method | effects | selection | structure |
| Genome editing via | +++ | +++ | + |
| CRISPR/Cas9 | | | |
| Overexpression of a construct | +++ | +++ | + |
| by plasmid transfection | | | |
| (integration) | | | |
| Overexpression of a construct | + | + | + |
| by plasmid transfection (no | | | |
| integration) | | | |
| Overexpression of a construct | ++ | +++ | ++ |
| by lentiviral transduction | | | |
| (genomic integration) | | | |

In one embodiment, the variant library is introduced into cell lines.

In another embodiment, the variant library consists of a library of gRNA delivered in combination with other necessary elements of CRISPR-Cas system by plasmid transfection or lentiviral transduction. In some cases, the cell to which the DNA construct is delivered, may be chemically treated or genetically modified to increase the probability that the genomic region targeted by the gRNA is successfully targeted.

One example of cell line which can be used to introduce variants of a variant library with genome editing via CRISPR/Cas9 is the human cell line HAP1. This is motivated by the following:
Substantial data supporting the use of HAP1 cells in MAVE-based variant classification, as disclosed, for example in Findlay et al. 2018 and Gasperini et al. CRISPR/Cas9-Mediated Scanning for Regulatory Elements Required for HPRT1 Expression via Thousands of Large, Programmed Genomic Deletions. AJHG 3; 101(2):192-205 (2017), the entireties of which are incorporated herein by reference.
HAP1 is a near-haploid cell line, meaning that it has only one copy of each gene that can be mutated with genome editing. As a result, haploidy improves the signal-to-noise ratio for the quantification of variant effects in comparison with diploid cells.
HAP1 is a well-characterized and frequently used cell line in biomedical research. HAP1 cells grow quickly in culture, which reduces the duration and cost of experiments.

Another example of cell line which can used are HEK293 cells, which is a well-characterized adherent diploid human cell line used frequently in biomedical research and which grow well in culture. Another example of cell line which can be used are K592 cells, which is a well-characterized triploid human cell lines which grows in suspension and is used frequently in biomedical research. This list of examples of cell lines which can be used is not exhaustive and other cell lines can be used depending on the case.

### Stratification of Variant Effects

FIGS. 11A and 11B, Step 3; Multiplex Functional Assay:

Genetic variation in the genomic region of interest should have clear molecular and/or cellular consequences (e.g., a marked effect on a molecular process such as a measurable alteration in RNA splicing or a marked effect on a cellular phenotype such as reduced cellular fitness). Alternatively, the region of interest should have clear clinical consequences when mutated (e.g., association with a highly penetrant monogenic disease, where every person with pathogenic variants in one or both copies of this gene will show a clinical phenotype), with an in vivo function that is easy to model or with easily identifiable surrogate markers. After introducing the variant library in cells, multiplex functional assays may be used to stratify genetic variants according to their effects on a measurable molecular and/or cellular phenotype. Alternatively, after introduction of the variant library, cells may be propagated through cell culture, processed and harvested for molecular and/or cellular analysis, through for example, DNA or RNA sequencing of single cells or of the cell population.

The functional assay used for variant stratification can be selected according to the function of the genomic region of interest. Assays measuring general molecular properties such as RNA levels, splicing, protein stability, protein localization and/or general cellular phenotypes such cell signaling or proliferation can be used. Additionally, specialized assays specific to the function of the region of interest can be selected (e.g., transcriptional output of a reporter gene when evaluating variants in regulatory sequences or the catalytic function of an enzyme when evaluating variants in such a protein-coding region). In this case, it is desirable that the assay specifically models the activities of a protein that are thought to be most relevant to its role in disease.

Biological assays which may be used for the multiplex functional assay include the following (but are not limited to these):
[gene expression/mRNA transcription]: saturation mutagenesis of regulatory elements (e.g., enhancers and promoters) to determine the effect of mutations on gene expression by quantification of transcript levels with single cell or bulk RNAseq.
[mRNA transcription/pre-processing]: saturation mutagenesis of coding regions within a gene to determine the effect of mutations on mRNA stability by quantification of transcript levels with single cell or bulk RNAseq.
[mRNA splicing]: saturation mutagenesis of coding and intronic regions within a gene to determine the effect of mutations on mRNA splicing by quantification of transcript isoforms with single cell or bulk RNAseq. This analysis is particularly important to detect the effect of mutations on tissue-specific transcript isoforms.
[protein abundance/stability]: saturation mutagenesis of coding regions within a gene to determine the effect of mutations on protein stability and abundance.
[protein function]: saturation mutagenesis of coding regions within a gene to determine the effect of mutations on the function of specific protein domains by quantification of protein function with a gene or protein-specific assay (e.g., PTEN lipid phosphatase assay).

The aim of the multiplex functional assay step is to stratify a cell population according to the effects of all genetic variants introduced in this cell population. In this case, a multiplex functional assay must be able to enrich, deplete or otherwise select the cells carrying mutations with the strongest effects from the population of cells. Alternatively, the aim of the multiplex functional assay step can be to measure in parallel the effect of all variants introduced in a cell population. The functional assay selected to test the variant effects should capture mechanisms through which pathogenic variants in the studied gene or genomic region cause human disease.

Generalizable and scalable assays are ideal to measure the effect of variants in different genes or genomic regions. For instance, massive parallel reporter assays can quantify the effect of variants in regulatory sequences throughout the genome (Melnikov, A. et al. Systematic dissection and optimization of inducible enhancers in human cells using a massively parallel reporter assay Nat. Biotechnol. 30, 271-277 (2012), the entirety of which is incorporated herein by reference). Similarly, assays examining general properties such as intra-cellular protein levels, protein location or cell fitness can be used to study genetic variation in protein-coding genes.

In some embodiments, highly scalable and automated platforms consisting of a battery of assays measuring general molecular properties (mRNA levels, splicing, protein levels, protein stability, protein localization) and cellular phenotypes (cell proliferation, cell signaling) may be used. Data may be collected from different cell types, to reflect the biological diversity of different tissues. Types of assays used in these platforms include:
Transcript abundance, stability and splicing: this assay is based on RNA sequencing of the sample to determine the ratio between RNA and DNA sequencing reads. This allows to estimate the effect of variants in regulatory regions including promoters, as well as the effect of variants in transcribed regions on mRNA transcript stability. Furthermore, splicing assays relying on RNA sequencing measure the effect of variants on transcript splicing (Julien, P., Miñana, B., Baeza-Centurion, P., Valcárcel, J. & Lehner, B. The complete local genotype-phenotype landscape for the alternative splicing of a human exon. Nat. Commun. 7, 11558 (2016), the entirety of which is incorporated herein by reference).
Gene expression: also relying on RNA sequencing, tests such as massively parallel reporter assays allow to measure the effect of variants in promoter and regulatory sequences on gene expression (Melnikov, A. et al. Systematic dissection and optimization of inducible enhancers in human cells using a massively parallel reporter assay. Nat. Biotechnol. 30, 271-277 (2012), the entirety of which is incorporated herein by reference).
Protein levels and protein stability: This assay is based on sorting for fluorescence of host cells expressing a protein with the variants of interest, which can either be tagged by one or more fluorescent proteins (e.g., GFP) or targeted by a fluorescent antibody. This assay separates cells with high and low levels of fluorescence, as an indirect measure of the protein levels and protein stability (Yen, H.-C. S., Xu, Q., Chou, D. M., Zhao, Z. & Elledge, S. J. Global Protein Stability Profiling in Mammalian Cells. Science (80-.). 322, 918-923 (2008); Matreyek, K. A. et al. Multiplex assessment of protein variant abundance by massively parallel sequencing. Nat. Genet. 50, 874-882 (2018), the entireties of which are incorporated herein by reference).
Protein function: Approximately 4000 human genes have been shown to contribute to the mechanisms underlying diseases and recent scientific publications estimate there are suitable multiplex functional assays to test the effects of genetic variants on protein function for ˜57% of them (Weile, J. et al. A framework for exhaustively mapping functional missense variants. Mol. Syst. Biol. 13, 957 (2017), the entirety of which is incorporated herein by reference).
Cell fitness as a measure of protein function: The most effective, generalizable and scalable assays currently available couple genetic variants to the fitness and growth of the host cell.
For instance, a functional (i.e., healthy) copy of the BRCA1 gene is essential for the survival and proliferation of individual cells from the HAP1 cell line. While HAP1 cells with neutral BRCA1 mutations grow well in culture, HAP1 cells carrying mutations that damage the function of BRCA1 die. A simple but effective multiplex functional assay to test the effect of BRCA1 variants on host cell fitness is to maintain the mutated cells in in vitro culture for 20 days. In a population of cells with different BRCA1 mutations growing in vitro, cells with damaging BRCA1 mutations are depleted and those with neutral mutations are enriched (see FIG. 14). Remarkably, this in vitro approach correctly identifies BRCA1 mutations that cause disease in humans (overall sensitivity of 97% and specificity of 98% in variant classification) (Findlay et al. 2018 as above).

In one embodiment, multiplex functional assays measuring cell growth in vitro which depletes damaging variants are used. The advantages of this are as follows:
Cost-effective and simple functional assay requiring minimal infrastructure and optimization.
According to recent large-scale studies, close to 30% of human disease genes lead to alterations in cell growth when mutated in vitro (Wang, T., Wei, J. J., Sabatini, D. M. & Lander, E. S. Genetic Screens in Human Cells Using the CRISPR-Cas9 System. Science (80-.). 343, 80-84 (2014); Blomen, V. A. et al. Gene essentiality and synthetic lethality in haploid human cells. Science (80-.). 350, 1092-1096 (2015); High-Resolution CRISPR Screens Reveal Fitness Genes and Genotype-Specific Cancer Liabilities. Cell 163, 1515-1526 (2015), the entireties of which are incorporated herein by reference). This supports that cell growth competition in vitro is a robust functional assay to identify damaging mutations for >1700 genes, including genes considered highly clinically significant (see Table 3 for more detailed examples).

Other examples of assays using cell fitness as a measure of the effect of a variant at the protein level include the following:
Pooled cell competition: In practice, this assay is similar to that described previously for cell growth with the important distinction that it enriches for damaging variants. Cells carrying variants with stronger effects on cell proliferation will outgrow those with variants with neutral or weaker effects. For example, cells with activating mutations in genes in the RAS/MAPK pathway proliferate faster than those with mutations that do not effect cell growth (Brenan, L. et al. Phenotypic Characterization of a Comprehensive Set of MAPK1/ERK2 Missense Mutants. Cell Rep. 17, 1171-1183 (2016), the entirety of which is incorporated herein by reference).
Drug response: These assays examine the effect of a drug on host cell fitness as an indirect measure of variant sensitivity or resistance to a drug. For instance, a pooled cell competition assay in the presence of a drug targeting the gene in which variants are tested selects cells with variants that are resistant to the drug. While relevant for cancer drugs, these assays can also be harnessed to examine the potential of response to drug treatment for patients harboring variants in genes not involved in cancer (e.g., response to ivacaftor in patients with cystic fibrosis due to mutations in CFTR with an unknown disease mechanism) (Wagenaar, T. R. et al. Resistance to vemurafenib resulting from a novel mutation in the BRAFV600E kinase domain. Pigment Cell Melanoma Res. 27, 124-133 (2014) and Giacomelli, A. O. et al. Mutational processes shape the landscape of TP53 mutations in human cancer. Nat. Genet. 50, 1381-1387 (2018), the entireties of which are incorporated herein by reference).
Synthetic rescue and synthetic lethality: These assays are based on the fact that, under otherwise lethal conditions, damaging variants in some genes enable survival of the host cell. For instance, exposure to 6-Thioguanine is lethal for wild-type cells. However, cells with damaging mutations in genes in the mismatch repair pathway survive exposure to this chemical (Zeng, X., Yan, T., Schupp, J. E., Seo, Y. & Kinsella, T. J. DNA Mismatch Repair Initiates 6-Thioguanine-Induced Autophagy through p53 Activation in Human Tumor Cells. Clin. Cancer Res. 13, 1315-1321 (2007), the entirety of which is incorporated herein by reference). Similarly, other multiplex functional assays are based on synthetic lethality, that is that, under certain conditions, damaging variants cause death of the host cell. (Mighell, T. L., Evans-Dutson, S. & O'Roak, B. J. A Saturation Mutagenesis Approach to Understanding PTEN Lipid Phosphatase Activity and Genotype-Phenotype Relationships. Am. J. Hum. Genet. 102, 943-955 (2018) the entirety of which is incorporated herein by reference)
Several additional assays examining cell fitness are well-established in other eukaryotic cell models. This includes, for instance, assays based on protein-protein interactions that enable the transcription of a reporter gene enabling host cell selection (Starita, L. M. et al. Massively Parallel Functional Analysis of BRCA1 RING Domain Variants. Genetics 200, 413-422 (2015); Bandaru, P. et al. Deconstruction of the Ras switching cycle through saturation mutagenesis. Elife 6, 1-31 (2017); Kim, I., Miller, C. R., Young, D. L. & Fields, S. High-throughput Analysis of in vivo Protein Stability. Mol. Cell. Proteomics 12, 3370-3378 (2013), the entireties of which are incorporated herein by reference).

Other examples of multiplex functional assays to assess the effect of genetic variants on individual protein function include the following:
Markers of protein function: this assay measures direct or indirect markers of protein function detected by fluorescence. It includes upregulation of gene expression as a consequence of protein function but can also examine enzymatic reactions, signaling cascades, receptor activity, cell cycle control, cell viability, protein-protein interactions and ion influx, among others.
Regulation of gene expression: this assay measures the effects of variants in regulatory regions controlling the expression of a protein-coding gene which can either be GFP-tagged or detected with a fluorescent antibody (Canver, M. C. et al. BCL11A enhancer dissection by Cas9-mediated in situ saturating mutagenesis. Doi:10.1038/nature15521, the entirety of which is incorporated herein by reference). This assay separates cells with high and low levels of fluorescence, as an indirect measurement of gene expression.

Table 5 offers examples on how different multiplex functional assays can be applied to clinically relevant genes. A current limitation for multiplex functional assays is that some genes with in vivo functions can be difficult to model in vitro and test in parallel. This includes genes involved in multicellular processes such as morphogenesis, neuronal migration and roles in cell junctions or extracellular matrix.

TABLE 5
Examples of clinically relevant genes and multiplex
functional assays to test variant effects.

| Example of genes | Type and disease category | Type of assay |
|---|---|---|
| APC, LDLR, SDHD, SDHC, | Diagnostic | Cell growth competition with |
| SDHB, NF2, VHL | Genetic disorders with | negative selection. |
| (genes from the American | established clinical | |
| College of Medical Genetics' | interventions. | |
| list of recommendations for | (e.g., increased risk for | |
| reporting of secondary | cancer, increased risk for | |
| findings in genetic testing) | cardiovascular events, | |
| | cardiac arrhythmias, etc . . .) | |
| BRCA2, PALB2, BARD1, | Diagnostic | Cell growth competition with |
| BRIP1, RAD51C, NBN, | Inherited cancer syndromes | negative selection. |
| MRE11A, RAD51D, CDK4, | with potential for early cancer | |
| RAD50, BAP1, FANCC, | detection (e.g., increased risk | |
| FANCD2, RAD51, FANCM, | of breast and ovarian cancer, | |
| XRCC2, ATR, BLM, BUB1B, | etc . . .) | |
| CDC73, CEP57, CHEK1, | | |
| ERCC2, ERCC3, ERCC4, | | |
| EZH2, FANCB, FANCL, FH, | | |
| KIT, MAX, SBDS, SLX4, | | |
| SMARCB1 | | |
| AFG3L2, COX10, COX15, | Diagnostic | Cell growth competition with |
| NDUFA9, OPA1, POLG2 | Mitochondrial diseases | negative selection. |
| (total of 80 nuclear-encoded | (e.g., blindness due to optic | |
| genes with mitochondrial | atrophy, movement disorders | |
| function) | due to spinal cerebral ataxia, | |
| | etc . . .) | |
| MSH2, MSH6, MLH1, PMS2 | Diagnostic | Synthetic rescue to 6- |
| (genes from the American | Hereditary cancer syndrome | thioguanine exposure. |
| College of Medical Genetics' | with potential for early cancer | |
| list of recommendations for | detection | |
| reporting of secondary | (i.e., Lynch syndrome, | |
| findings in genetic testing) | predisposing to cancer at a | |
| | young age) | |
| All clinically relevant protein- | Diagnostic | Fluorescence-based cell |
| coding genes | All hereditary disorders | sorting to measure protein |
| (~4,000 genes) | | levels and protein stability. |
| | | RNA sequencing to |
| | | determine transcript |
| | | abundance, stability and |
| | | splicing. |
| AKT1, AKT3, BRAF, HRAS, | Diagnostic | Cell growth competition with |
| KRAS, MAP2K1, MAP2K2, | RASopathies | positive selection |
| MAP2K3, MAPK1, MAPK3, | (i.e., congenital disorders | |
| MAPK7, NF1, NRAS, SOS1, | affecting 1 in 1000 children) | |
| SOS2 | | |
| (200 genes in the RAS/MAPK | Future potential for drug | |
| pathway) | matching for these patients. | |
| | Drug matching | Drug response in cell growth |
| | Sensitivity to drug treatment | competition assay with |
| | for sporadic cancer | positive selection |

This is by no means an exhaustive list of possible multiplex functional assays (Starita, L. M. et al. Variant Interpretation: Functional Assays to the Rescue. Am. J. Hum. Genet. 101, 315-325 (2017), the entirety of which is incorporated herein by reference).

### FIGS. 11A and 11B, Step 4; Sequencing to Quantify Variants in Cell Population:

The aim of this step is to quantify the number and composition of variants in the studied cell population, allowing for a comparison before and after the functional assay.

Samples from the population of cells are harvested before and after the functional assay. Then, genetic material, such as DNA or RNA, from these cells is extracted, processed and sequenced on a Next Generation Sequencing platform, such as an Illumina sequencing platform (e.g., MiSeq, HiSeq, NovaSeq, etc.). The composition and frequency of variants or variant barcodes in the sequencing data reflects the composition and frequency of variants in the cell population (see FIG. 15). This allows measurement of variant effects from the sequencing data, by examining enrichment and depletion of variants in the cell population before and after the functional assay. Established disease-causing variants, loss-of-function variants and synonymous variants can be used to calibrate the analysis.

The end product of a MAVE experiment is a high-quality data set detailing the measurement of the effects of hundreds or thousands of variants in different biological processes. The measurements of variant effects at the molecular and/or cellular level can be included in several ways as part of the input data used for training the Bayesian Network for variant classification.

First, the molecular and/or cellular measurements of variant effects are used directly to support the novel classification of genetic variants. This can be done following a manual or automated process of variant classification according to the guidelines from the American College of Medical Genetics, as part of the criteria for functional evidence demonstrating that a variant has abnormal or normal gene and/or protein function (PS3/BS3 criterion). This allows for new genetic variants to be classified as benign and/or damaging, thus increasing the number and unbiasing the data sets of classified (i.e., labelled) genetic variants that can then be used to train machine learning models.

Second, in another embodiment, the molecular and/or cellular measurements of variant effects derived from MAVE experiments can be processed and normalized to be annotated and used directly as features input for one of the nodes of the Bayesian Network for variant classification (i.e., as for instance, other annotated features included such as measures of evolutionary conservation). The molecular and/or cellular measurements of variant effects derived from MAVE experiments can be used as features within the training data set provided to the variant classification method of the embodiment. During classification of a patient's genomic variants, individual genomic variants of the patient are, in one embodiment, annotated with the relevant data derived from MAVE experiments to form the input features provided to the variant classification method of the embodiment.

Third, as discussed above, the molecular and/or cellular effects of genetic variants can be processed and used to train an intermediate specialized machine learning model, from which predictions for molecular and/or functional scores are generated, which in turn are used as a feature in the input data to train the hierarchical Bayesian network for variant classification. For example, if the biological experimentation carried out consists in measuring the effects of genetic variants on mRNA levels, these measurements can be processed and used as part of the input training data for a machine learning algorithm specialized in predicting the effects of genetic variants on mRNA levels. Here, the measurements obtained from biological experiments can be used as a predicted label or score for the effect of genetic variants on mRNA splicing. After training, the specialized machine learning algorithm then imputes the effect of unobserved genetic variants on mRNA levels as a label or score. In an embodiment, these predicted scores or labels are then used as input data for the hierarchical Bayesian network for variant classification, representing a feature to be used as input in one of the nodes of the network.

### Sequencing Library Preparation

Depending on the type of experimental assay carried out, samples from the population of studied cells can be harvested after the delivery of the variant library but prior to the functional assay, in order to provide a baseline measure of the composition of the cell population. Samples from the population of studied cells can be harvested after the functional assay, to measure changes in the composition of the cell population after cell selection using the functional assay. Alternatively, individual cells from the population of studied cells can be harvested and/or processed individually to measure the effects of the genetic alterations resulting from the introduction of the variant library. Genetic material from these cells, such as DNA or RNA is isolated using methods such as manual or automated extraction with spin columns or organic extraction using chemical compounds.

The extracted DNA or RNA is then processed to generate a sequencing library that can be sequenced on a high throughput sequencing platform, such as an Illumina sequencing platform as MiSeq, HiSeq or NovaSeq. A genetic or genomic region of interest, such as the genomic locus in which genetic variants were introduced, is amplified and barcoded by adding unique molecular identifiers (UMIs) by polymerase chain reaction (PCR). Alternatively, elements of the variant library introduced in the cells, such as the variant library itself or barcodes serving as identifiers specific to the variant introduced, can be amplified from the DNA or RNA extracted from the cells and barcoded by PCR. Sample barcodes and sequencing adaptors are then added to the amplified DNA and RNA fragments by PCR and adaptor ligation. Alternatively, a sequencing library preparation kit can be used to prepare the extracted DNA or RNA for sequencing on a sequencing platform.

A sample of the variant library is also subjected to sequencing library preparation by amplification, barcoding and adaptor ligation for subsequent sequencing, in order to measure the composition of the variant library prior to being introduced in the cells. This allows to determine whether all genetic variants designed are represented in the library and serves as a quality control step.

The sequencing libraries generated are then submitted for quality control. The libraries are visualized using an electrophoresis system such as an agarose gel, microfluidics-based electrophoresis or capillary electrophoresis. The concentration of the sequencing library is determined with a highly sensitive method, such as fluorometry measurements with a Qubit system. After passing quality controls and the determination of the concentration, the sequencing libraries are diluted to desired concentrations and can be pooled with other samples for multiplex sequencing.

### Sequencing

After processing, the sequencing libraries are sequenced on a high throughput sequencing platform with low error rates, such as an Illumina platform as MiSeq, HiSeq, NovaSeq, etc. The libraries can be sequenced on different types of sequencing runs (e.g., single end read or paired-end reads) with variable read length (e.g., 100 bp single reads, 100 bp paired-end read length, 150 bp paired-end read length, etc.). Sequencing of the libraries can take place at variable sequencing depths, according to the expected complexity of the library and the number of cells used in the experiments. For example, sequencing of the libraries may be run on a MiSeq run with paired-end 150 bp read length, at a sequencing depth of 1 million reads for the variant library, 9 million reads for the baseline sample and 15 million reads for the treated sample. Alternatively, sequencing of the libraries may be included as part of a larger sequencing run on a HiSeq run with paired-end 150 bp read length, at a sequencing depth of 2 million reads for the variant library, 20 million reads for the baseline sample and 40 million reads for the treated sample.

Alternatively, sequencing can be carried out in other sequencing platforms enabling long reads, such as Nanopore technologies or Pacific Biosciences technologies. In this case, multiple pass sequencing and sequencing error correction may be necessary to correctly identify individual genetic variants and/or variant barcodes sequenced.

### Data Processing

The data generated by high-throughput sequencing is processed following standard bioinformatics pipelines. The raw sequencing data generated by the sequencer is output into BCL files, which can be processed and demultiplexed into an individual FASTQ file per sample sequenced, which can be used for downstream analysis. FASTQ files can be processed with different existing pipelines, such as samtools, to determine the number of sequencing reads carrying individual genetic variants of interest in a sample. For example, counts of the number of reads carrying individual genetic variants per sample can be determined using approaches such as Compact Idiosyncratic Gapped Alignment Report (CIGAR) strings or alternatively, pileups can be generated to determine the presence and composition of genetic variants for each individual base pair for each sample. The output of this step is a count of the number of sequencing reads for each individual genetic variant, as a proxy of the number of cells in which the genetic variant is present in the sample. This allows to compare changes in genetic composition of the sample as a consequence of the functional assay used to stratify genetic variants for their effects.

If UMIs are used to barcode individual molecules, an additional step can take place in which sequenced molecules carrying UMIs observed more than once in a specific sample are collapsed, thus removing duplicate observations from the sequencing data generated (and thereby providing a more accurate measurement of the number of genetic variants in a sample by sequencing).

### Data Analysis

After sequencing and initial data processing, individual mutation measurements are derived from the sequencing data for each genetic variant within the variant library. These mutation measurements reflect the effect of the genetic variant on a specific molecular and/or cellular property being quantified through the multiplex functional assay (e.g., effect of the genetic variant on cell growth or on protein stability). The measurement of molecular and/or functional effects is derived by quantifying the level of enrichment or depletion that the multiplex functional assay had over cells harboring each individual genetic variant.

Depending on the type of functional assay used to stratify the effects of mutations, the measurement of molecular and/or functional effects can be determined by comparing the individual genetic variant counts in the sample before and after the functional assay. Alternatively, the measurement of mutation molecular and/or functional effects can be determined by binning genetic variants according to their effects. Loss-of-function variants, synonymous variants and known damaging or disease-causing variants can be used to calibrate the analysis. Generally speaking, loss-of-function variants such as stop mutations are the most damaging to the function of a gene or its gene product, while synonymous variants have little or no effect on the function of a gene or its gene product. As such, the measurements of the molecular and/or cellular effects of these types of genetic variants can be used first, to determine the range of molecular and/or cellular effects captured by the assay and second, to normalize the measurements obtained for other variants and compare results across experiments. Furthermore, known damaging and/or disease-causing genetic variants can be included as part of the library of genetic variants to be evaluated in the experiment, to determine the sensitivity and specificity of the assay in detecting damaging or disease-causing genetic variants.

The molecular and/or cellular measurements of variant effects can be used directly to support the novel classification of genetic variants according to guidelines from the American College of Medical Genetics, as part of the criteria for functional evidence demonstrating that a variant has abnormal or normal gene and/or protein function (PS3/BS3 criterion). This allows for new genetic variants to be classified as benign and/or damaging, thus increasing the number and decreasing the bias in data sets of classified (i.e., labelled) genetic variants.

The molecular and/or cellular measurements of variant effects can be processed and normalized to be used directly as features within the training data set, serving as input for one of the nodes of the Bayesian Network for variant classification (i.e., as for instance, other features included such as measures of evolutionary conservation).

The molecular and/or cellular effects of genetic variants can be processed and used to train an intermediate specialized machine learning model, from which predictions for molecular and/or functional scores are generated, which in turn are used as a feature in the input data to train the Bayesian Network for variant classification. For example, if the biological experimentation carried out consists in measuring the effects of genetic variants on mRNA levels, these measurements can be processed and used as part of the input training data for a machine learning algorithm specialized predicting the effects of genetic variants on mRNA levels. Here, the measurements obtained from biological experiments can be used as a predicted label or score for the effect of genetic variants on mRNA splicing. After training, the specialized machine learning algorithm can then predict the effect of unobserved genetic variants on mRNA levels as a label or score. These predicted scores or labels can then be used as input data for the Bayesian Network for variant classification, representing a feature to be used as input in one of the nodes of the network.

### A Cybernetic Loop Created by Combining Bayesian Networks & Biological Experimentation

Existing approaches to MAVEs often use assays that are specific to particular genes or gene functions (e.g., BRCA1 homology directed repair activity or PTEN lipid phosphatase activity). Due to the specificity of these assays, both the scalability of the experimental approaches and the generalizability of the data generated are limited. On one hand, gene- or gene function-specific experimental assays may require extensive fine-tuning of experimental conditions to be used for other genes or genomic regions (or in some cases, simply cannot be used). On the other hand, data generated using gene-specific assays may be measuring the consequences of genetic variants on a specific molecular property that cannot be extrapolated to genetic variants in other genes. In contrast, assays quantifying general molecular properties (such as mRNA transcription, mRNA splicing, protein translation, protein stability and/or protein function) can be scaled to genes and/or genomic regions throughout the genome. Furthermore, the data generated through this approach reflects general molecular properties that can be extrapolated to other genes or genomic regions. Through this process of generalization, insights into biological processes can be obtained.

The scalability of the assays and generalizability of the biological data generated enable a cybernetic loop, which combines the generation of biological data through high-throughput experiments (such as MAVEs) with the hierarchical Bayesian network for variant classification. Essentially, it functions as a feedback circuit between the predictions generated by an algorithm and the empirical validation of these predictions in the laboratory, where the model predictions guide the generation of biological data and the biological data generated is used to improve model predictions.

More specifically, this cybernetic loop of an embodiment consists of the following: first, the hierarchical Bayesian network for variant classification generates predictions for a list of genetic variants, such as potential molecular, cellular and/or clinical consequences, together with a posterior probability reflecting the evidence supporting this prediction and a confidence score. Then, genetic variants for which the predicted effects are given with low probability or low confidence due to missing or conflicting evidence are selected for further investigation using MAVEs or other high throughput experiments. As assays measuring general molecular properties can be scaled to genes and/or genomic regions throughout the genome, new biological data is generated experimentally for the genetic variants of interest. This data consists of measurements of molecular and/or cellular consequences of the genetic variants (such as effects on mRNA transcription, mRNA splicing, protein translation, protein stability and/or protein function). The biological data can be analyzed to generate measurements and/or scores for the effects of a genetic variant on a specific molecular and/or cellular process, which are incorporated in the data used to train the variant classification algorithm. Because the biological data reflects general molecular and/or cellular properties of a genetic variant, underlying patterns can be generalized to genetic variants in other genes or genetic regions with similar properties (e.g., if one variant with specific properties is damaging to protein stability, other variants with similar properties may also be damaging to protein stability). This process therefore offers insights, on one hand, into the reasoning process of the variant classification algorithm and, on the other, potentially into the molecular mechanisms of genetic variant effects. Finally, this process improves the performance of the model by avoiding overfitting to the data and constraining the parameter search space.

### III. A Hierarchical Bayesian Network for Variant Prioritization

Variant prioritization is the process of ranking a list of candidate pathogenic genetic variants identified in a patient sample, to provide a molecular and clinical diagnosis for a patient with a suspected genetic disease. Variant prioritization involves estimating through statistical methods the probability of a patient having a particular genetic disease, given their specific clinical features and one or more specific candidate pathogenic genetic variants.

A patient's clinical features are presented as a list of terms describing clinical signs, symptoms and/or phenotypic abnormalities observed in the patients (such as a list of terms from the Human Phenotype Ontology (HPO) or other ontology systems).

Additionally, quantitative physiological and/or phenotypic data can be submitted as part of the terms describing the clinical abnormalities encountered in the patient. Candidate disease-causing genetic variants can be obtained by classifying a list of genetic variants identified in the patient's DNA sample (as described above).

### Existing Algorithms for Variant Prioritization and Limitations

Previous computational approaches to variant prioritization have made an effort to automate this process using different methodologies (e.g., Phenomiser, Exomiser, Phevor, etc. . . . ). Some variant prioritization approaches rank genetic variants by comparing a given patient's clinical features against clinical features of known genetic disorders to find potential matches (i.e., calculating phenotype similarity). If the patient's clinical features are highly similar to those of a known genetic disease, then genetic variants in the gene associated with this disease are prioritized (i.e., ranked as more likely to cause the patient's clinical features). However, this approach is computationally intensive and limited in that only patients with known genetic disorders (i.e., having disease-causing genetic variants in genes known to be associated with a specific genetic disease) can be diagnosed. To circumvent this issue, other methods have used alternative approaches to variant prioritization enabling novel disease-gene associations (either as standalone or in combination with phenotype similarity approaches). One such approach to variant prioritization is to rank genetic variants by gene similarity between the gene in which the candidate variant is found and other genes known to be implicated in genetic diseases. Gene similarity can be calculated in several ways, for instance by linking gene similarity back to phenotype similarity using the HPO graph (i.e., two genes with similar phenotypes are more similar than two genes with different phenotypes) or by calculating distance between two genes in random walks within gene or protein-protein interaction networks. However, using phenotype similarity to estimate gene similarity has limitations and random walks are computationally intensive, which hamper the performance of these variant prioritization algorithms.

Advantages of Hierarchical Bayesian Networks in Combination with Computational Geometry for Variant Prioritization

In contrast with existing methods, the machine learning approach disclosed herein involving hierarchical Bayesian networks in combination with computational geometry provides a better approach to the task of variant prioritization due to the following:
1. Ability of the Bayesian framework to quantify evidence in support of the null hypothesis: As discussed previously, the Bayesian statistical framework provides the unique ability to quantify how much the data supports alternative hypotheses (e.g., whether the combination of a specific genotype and phenotype is an indication of a specific genetic disease or not). This additionally enables the algorithm to deal with conflicting or inconsistent evidence during training and when making predictions, which is vital in diagnostic reasoning since it allows for the computation of posterior probabilities and confidence scores around the predictions, which take into account various aspects of the data.
2. Ability of hierarchical Bayesian networks to capture complex causal relationships spanning multiple factors: Graphical structures, such as hierarchical Bayesian networks, can intuitively represent complex relationships between cause and effect. As such, graph models are ideal mathematical tools to compute probabilities in the context of hierarchical causal dependencies such as the association between damaging genetic variants in a specific gene, a series of clinical features and a specific genetic disease.
3. By enabling the former principles, hierarchical Bayesian networks allow for fast and efficient computation of posterior probabilities: The graphical structure of the model allows to specify how the features combine together when expressing a probability model (i.e., the probability of observing a disease given a phenotype and genotype signature vectors). In contrast with other statistical methods from the Bayesian framework, Bayesian networks restrict the search space, allowing for fast and efficient computation of posterior probabilities for combinations of genetic variants, clinical features and genetic disease.
4. The combination of hierarchical Bayesian network with computational geometry enables the simultaneous evaluation of the similarity of associations between phenotype, genotype and disease: In contrast to calculating similarity scores which measure the distance between two terms in an ontology graph (e.g., such as the HPO graph), the use of computational geometry allows the calculation of similarity scores that measure distances in an arbitrary n-dimensional space, recasting the issue of comparing similarities among different phenotype-genotype-disease associations in a more general and computationally efficient framework (e.g., distances in a 3-dimensional space). This is of particular interest for estimating gene similarity, as it can be achieved in a manner that is independent from the phenotype and the HPO graph (see above). A genotype or phenotype is defined as a set of coordinates in geometric space. The similarity between two genotypes or two phenotypes is calculated by measuring the distance between these two sets of coordinates. The use of computational geometry to calculate genotype similarity in combination with the use of hierarchical Bayesian networks offers unprecedented advantages in analyzing highly dimensional data sets, as the machine learning model can be trained to extract knowledge from the entire data space (e.g., how change in one dimension affects each of the other dimensions) rather than single data points.
   IIIa. Description of the Hierarchical Bayesian Network Used for Variant Prioritization

In one embodiment, variant prioritization step 120 of the method shown in FIG. 2 is implemented through a second, separate hierarchical Bayesian network for variant prioritization. The hierarchical Bayesian network model is defined by:
a. the set of variables/nodes (either observed or hidden), each with its own parameters (conditional probability distributions or CPDs)
b. the causal dependencies linking the nodes (variables) expressed as directed edges in the graph structure.

Being cast in a Bayesian statistical framework, the variant prioritization process inherits the previously mentioned benefits.

The input for process 120 is one or more of:
a patient's genetic data in the form of a VCF file containing a list of candidate disease-causing genetic variants identified in the patient's DNA sample;
an output of a classification algorithm, such as, in one example, the output of one of the classification algorithms described herein, including optionally also an indication of the confidence in the classification (e.g., the output of process 110, comprising a list of classified genetic variants with the posterior probability of each classification);
a patient's phenotype data in the form of a list of terms describing the patient's clinical features (e.g., from the Human Phenotype Ontology (HPO) or other ontology systems).

The output of process 120 is a list of most likely molecular and clinical diagnosis for the patient, based on a ranked list of posterior probabilities for associations between different diseases and the genotype and phenotype observed in the patient. The process of automated variant prioritization 120 is illustrated in more detail in FIG. 16.

During both training of the network and use for prioritization of genetic variants in a patient, genetic and/or phenotype data is processed using a pipeline for signature extraction prior to entering the hierarchical Bayesian network for variant prioritization. FIG. 17 illustrates the format of input data for the hierarchical Bayesian network for variant prioritization, after pre-processing (i.e., after signature extraction) has been completed. The data provided in the table shown in FIG. 17 are discussed in more detail below.

FIG. 18 illustrates the process through which the genotype and/or phenotype data are processed to derive genotype and/or phenotype signatures, expressed as geometric coordinates that serve as input to the prioritization algorithm. A patient's genotype data comprises a plurality of genotype data points. Each genotype data point in turn comprises information relating to the genetic variant in question identified in the patient sample. A patient's phenotype data comprises a plurality of phenotype data points. Each phenotype data point in turn comprises information relating to one or more clinical features of the patient.

During signature extraction, the patient genotype and phenotype data is submitted and processed along with a reference data set for genotype and phenotype data for all known human genetic diseases. These reference data sets allow for the patient's genotype and phenotype data to undergo dimensionality reduction and projection in n-dimensional space, within the context of known human genetic diseases. It will be appreciated that these reference data sets remain the same during training of the classification network as well as during its production mode in which genetic variants of patients are classified.

Reference genotype data is a reference data set that comprises a plurality and ideally all known genetic variants that have been characterized as disease causing. It consists of a data set or a subset of a data set containing a list of all genetic variants classified as disease causing in known genetic diseases in humans (i.e., all known genotypes associated with disease) and is derived from curated datasets (e.g., public databases such as ClinVar) and published literature. Each genotype data point comprises information relating to a genetic variant in question identified as disease-causing.

Reference phenotype data is a reference data set of known clinical features associated with disease. It consists of a data set or a subset of a data set containing the clinical phenotype of all known genetic diseases in humans (i.e., a list of clinical terms describing each of all known genetic diseases) and is derived from curated datasets (e.g., public databases such as Orphanet or OMIM) and published literature. Each phenotype data point comprises information relating to one or more clinical features associated with a specific genetic disease. It will be noted that the reference genotype and phenotype data are not necessarily linked for signature extraction and can be processed independently from each other in this step. The reference genotype data is submitted as a list of genetic variants in a VCF file. The reference phenotype data is submitted as lists of one or more terms describing the clinical phenotype associated with individual genetic diseases. The patient's genotype and phenotype data is input to the signature extraction pipeline along with the reference genotype data and reference phenotype data.

After reading the list of genetic variants submitted in the VCF file and/or the list of clinical phenotype terms submitted, the automated pipeline maps each genetic variant and phenotype term to a corresponding ontology (e.g., Gene Ontology, Variation Ontology or a protein-protein interaction network for the genotype; Human Phenotype Ontology for the phenotype). For the genetic variant, this is carried out by matching the label of the gene affected by the genetic variant (i.e., the gene id or gene symbol) to the corresponding label of the gene in the selected ontology and/or the terms within the ontology associated with this gene label. For example, a patient is found to carry a pathogenic variant in the SETBP1 gene, which matches the SETBP1 gene in the Gene Ontology and is associated with the terms “protein binding” and “DNA binding,” among others. Alternatively, mapping each genetic variant to the corresponding ontology can be carried out by mapping the genetic variant to the corresponding label of the genetic variant in the selected ontology and/or terms associated with this genetic variant within the ontology. For the phenotype, the terms used to describe the clinical features found in a patient or associated with a genetic disease are matched to the exact term within the ontology selected. For example, a patient is found to have neurodevelopmental delay, which matches HPO term “HP:0012758—Neurodevelopmental delay” in the HPO graph. In particular in a first step in FIG. 18, each data point of the input vector formed by the genotype or the phenotype data is mapped back to a relevant ontology to identify the data point's location within the ontology graph (e.g., the Gene Ontology or to a protein-protein interaction network). An input data point in the patient's genotype is hereby a determined genetic variant. Mapping the terms back to an ontology graph establishes the position of a term within the ontology and its relationship to the rest of the terms within the ontology. The ontology in itself contains semantic information as to how different terms are connected and how closely connected they are. The distance between terms across the ontology can be considered a measure of the similarity of the terms. The mapping of the gene or genetic variant to the ontology consequently allows to determine a location of the gene or genetic variant and/or of the terms associated with this gene or genetic variant within the ontology. This location of the gene or genetic variant and/or its associated terms within the ontology is quantified by individually pairing the gene or genetic variant and/or its associated terms within the ontology with a plurality of gene or genetic variants and/or their associated terms mapped to the ontology or indeed individually with every gene or genetic variant and/or their associated terms mapped to the ontology. This location of the gene or genetic variant and/or its associated terms within the ontology reflect the relationship between a gene or genetic variant and/or their properties with other genes or genetic variants and/or their properties as described within the ontology. The pipeline subsequently computes an individual similarity score for all possible pairings of a submitted genotype data point with these entries in the ontology. This is shown as the second step in FIG. 18. To calculate this similarity score between an input data point and an entry of the ontology the similarity score between the respective point in the ontology to which the input data point has been matched is calculated. In one embodiment, the similarity between individual genes or genetic variants is computed as the semantic distance between terms in the corresponding ontology graph (e.g., gene ontology graph). A standard metric which incorporates graph information (e.g., shortest path length between two terms or minimum common ancestors (e.g., LIN, resNIK, PHENsim, mEUD) can be used for this purpose. In the third step shown in FIG. 18, all pair-wise similarity scores obtained previously are organized in a multidimensional matrix of genotype similarities (i.e., representation similarity matrix).

Additionally the pipeline computes an individual similarity score for each pairing of the submitted phenotype data points (each data point is, in one example, a list of HPO terms describing the clinical phenotype observed in a patient or associated with a known genetic disease) in the same manner as for the genotype data.

In the third step shown in FIG. 18, all pair-wise similarity scores obtained previously are organized in a multidimensional matrix of genotype similarities (i.e., representation similarity matrix), generating a m×m representational genotype similarity matrix, wherein m is the number of genotype input data points. The same step is also performed for the calculated phenotype similarity scores, generating a representational phenotype similarity matrix.

In the fourth and final step shown in FIG. 18, the pipeline applies multidimensional scaling (MDS) to each matrix to generate the principal components of each matrix to be used subsequently in the prioritization step as the genotype and/or phenotype signatures respectively. These are expressed as a set of geometric coordinates in n-dimensional space for the genotype and phenotype data submitted (i.e., one set of geometric coordinates per genetic variant and per list of terms describing a clinical phenotype), which are then used as input for the prioritization algorithm. In the example data shown in FIG. 17, the dimensionality of each of the representational genotype matrix and the representational phenotype matrix has been reduced to n=3, providing the genotype and phenotype signatures shown in FIG. 17. It will be appreciated that the phenotype and genotype signature do not have to have but can have the same dimensionality. Additionally, the principal components of the genotype and/or phenotype signatures can be visualized to provide diagnostic summary statistics useful to evaluate the quality of the data structures (e.g., signal to noise ratio, outliers).

During training of the hierarchical Bayesian network for variant prioritization, the input data consists of associations between disease-causing genetic variants and clinical features (e.g., one or more associated HPO terms) with a genetic disease identified by a disease identification number (e.g., OMIM id) as shown in FIG. 17. Alternatively, the input data during training may consist of gene-disease-phenotype maps (i.e., lists of genes or genetic variants associated with specific genetic diseases and specific clinical features). During use of the fully trained prioritization algorithm, the input data comprise a list of candidate disease-causing genetic variants identified in the patient (e.g., a list of classified genetic variants, such as the output from process 110) and/or a list of terms describing their clinical features (e.g., HPO terms). During use of the fully trained algorithm, the reference genotype and phenotype data is used only for signature extraction and not included as input data to the prioritization algorithm.

The input data shown in FIG. 17 comprise a data matrix with a number of rows and columns. In the embodiment, each row relates to the association between a genetic variant and a list of clinical features (e.g., one or more associated HPO terms), with the columns providing further details on the genetic variant, the clinical phenotype or their association with a genetic disease. The data matrix contains columns for the genotype signatures (the columns labelled “gen_coord1,” “gen_coord2” and “gen_coord3” together form the genotype signature in the example shown in FIG. 17) and phenotype signatures (the columns labelled “phen_coord1,” “phen_coord2” and “phen_coord3” together form the phenotype signature in the example shown in FIG. 17) of the genetic variant and the clinical phenotype in question. These signatures have been calculated in the manner described above.

Whilst in FIG. 17 the genotype and phenotype signatures respectively are formed by merely three coordinates/columns, it will be appreciated that embodiments are not limited in this manner. Instead n columns for n geometric coordinates in a n-dimensional space may be provided as input signature. It will moreover be appreciated that the genotype signature and the phenotype signature may have but do not have to have the same dimensionality. When the genetic variant and clinical features are associated with a known genetic disease, the disease id (e.g., OMIM disease id) is provided in an additional column (the column “disease_ID” in FIG. 20 provides the ID of the disease associated with the genotype and the phenotype signature of the row in question). It will be appreciated that it is the identification of the disease that is ultimately desired to be provided by the prioritization algorithm and that consequently the disease_ID is not provided in input data during “production mode” of the trained hierarchical Bayesian network.

As an example, three coordinates respectively are provided in FIG. 17 both for the genotype data and the phenotype data, in addition to the disease id associated with the genotype and phenotype. As a further example, the dotted box shown in FIG. 17 contains additional genotype information, that is the classification of the genetic variant in questions and the confidence score for this classification, which can be used as input for nodes 1650. The machine learning algorithm for variant prioritization is a hierarchical Bayesian network comprising observed nodes (described in more detail below) that accept the input data as shown in FIG. 17. It will be appreciated that during regular use of the trained variant prioritization network, the input data vectors may be incomplete (e.g., most frequently, due to missing information on the disease presented by the patient analyzed, but alternatively due to missing genotype or phenotype data).

A high-level example of the machine learning model used in an embodiment, for genetic variant prioritization is shown in FIG. 19.

Input 1610 is configured to receive the input data described above with reference to FIG. 17. During training of the prioritization algorithm, the minimum input data consists of a list of genotype-phenotype-disease associations, each of which is represented by: 1) a genetic disease id; 2) a genotype signature, expressed as a set of geometric coordinates corresponding to a single genetic variant or gene associated with the genetic disease as described above; and 3) a phenotype signature, expressed as a set of geometric coordinates corresponding to a list of clinical features associated with the genetic disease also as described above. Additional genotype (e.g., variant classifications, posterior probability) or phenotype data (e.g., qualitative or quantitative physiological and/or phenotypic data describing clinical abnormalities encountered in the patient) can be provided as input data to the network during training. During training of the network, genotype and phenotype data relating to a specific genetic disease (an extract of an example is shown in FIG. 17) is applied to the network 1600/input 1610, one row at a time (i.e., one genetic variant-phenotype-disease association at a time).

During use of the fully trained prioritization algorithm, the input data for each patient analyzed minimally consists of genotype data, represented by a list of genotype signatures (i.e., geometric coordinates, with one set of coordinates per genetic variant submitted) and/or phenotype data, represented by a single phenotype signature (i.e., one set of geometric coordinates, corresponding to the list of clinical features submitted for the patient). Additional genotype (e.g., variant classifications, posterior probability) or phenotype data (e.g., qualitative or quantitative physiological and/or phenotypic data describing clinical abnormalities encountered in the patient) can be provided as input data to the network during regular use of a trained network. During use of the fully trained prioritization algorithm, genotype and phenotype data relating to a patient is applied to the network 1600/input 1610, one row at a time (i.e., one genetic variant-phenotype association at a time).

The observed nodes of the network receiving the input data can be connected with one or more additional nodes, which can be themselves observed or hidden. Observed nodes are nodes for which there is input data available, reflecting direct empirical observations or measurements of a variable. Hidden nodes are nodes for which no input data are available because the variable which is captured by the node is not directly observable nor measurable. The connection between two nodes indicates causal dependency, with the parent node bearing causal influence on the child node. Parent-child node relationship is indicated by the directionality of the arrow in the graphic representation of the network. Observed nodes can therefore be causally dependent of one or more parent observed and/or hidden nodes. Hidden nodes can equally be causally dependent of one or more parent observed and/or hidden nodes. Observed and hidden nodes may adopt various discrete states; alternatively, nodes representing suitable properties (e.g., geometric coordinates representing genotype or phenotype signatures) may be able to adopt any particular value within a continuous range.

The connection between two nodes represents the statistical dependency between these two nodes, including the directionality of the causal influence. The statistical dependency between two nodes is captured by the flow of information from parent node to child node: first, evidence is provided to the parent node, which computes the probability of observing this evidence. Then, the evidence computed by the parent node flows to the child node, which computes the probability of each of the possible states in the child node, given the information provided by the parent node. If the child node has more than one parent node, the evidence from all parent nodes flows to the child node, to compute the probability of each of its possible states. If no evidence is directly input to a node, the node computes the probability of each of its possible states on the basis of available evidence from its parent nodes. The state with the highest probability is retained as the state of this node. Inference on the state of a child node can therefore be done on the basis of evidence in any of its parent nodes. Equally, inference on the state of any parent node can be done on the basis of evidence in any of its children nodes. Furthermore, inferences can be propagated throughout the system along any path within it (i.e., belief propagation).

The network 1600 is configured to predict the likelihood of combinations of genotype data (including genotype signatures as a proxy for the properties of a genetic variant observed in the patient) and phenotype data (including phenotype signatures as a proxy for the clinical features observed in the patient) being indicative of a specific genetic disorder. Based on this information, the output 1680 of the network 1600 is a list of candidate genetic diseases presented by the patient, and for each genetic disease, the probability that this is the disease presented by the patient, given a specific genetic variant and the clinical features observed in this individual.

The network 1600 separates nodes into those nodes 1620 that relate to the genotype data and those nodes 1630 that relate to the phenotype data under consideration. More specifically, the nodes 1620 comprise one or more nodes 1640 that relate to the genotype signature (i.e., the geometric coordinates in n-dimensional space of each genetic variant) and one or more nodes 1650 that relate to additional genotype characteristics (i.e., classification of the genetic variant, posterior probability of the classification).

Node 1640 represents the projection of each genetic variant in a n-dimensional geometric space, in which each genetic variant processed is identified by n-coordinates specifying its exact location in this n-dimensional space. As discussed, this n-dimensional geometric space is an abstract representation of all genetic variants, defined by biologically or clinically relevant relationships between them (e.g., as represented in Gene Ontology or through biological interaction networks). This n-dimensional geometric space is obtained by performing metric-preserving dimensionality reduction (e.g., multi-dimensional scaling) to the matrix of similarity scores calculated for all genetic variants being processed. Node 1680 represents genetic diseases, caused by one or more genetic variants and manifesting clinically with one or more clinical phenotypes. The connection between node 1640 and node 1680 represents the statistical dependency between the genetic variant being processed and a genetic disease potentially caused by this genetic variant. Node 1640 receives input data pertaining to the genotype signature of a genetic variant being processed. Based on this, node 1640 calculates the probability of observing this data and propagates the state with the highest probability of node 1640 this value to node 1680.

Node 1650 represents additional information pertaining to the genetic variant being processed (such as the posterior probability of the classification, output from process 110). The connection between node 1650 and node 1680 represents the statistical dependency between additional information pertaining to the genetic variant being processed and a genetic disease potentially caused by this genetic variant. Node 1650 receives input data pertaining to the genetic variant being processed. Based on this, node 1650 calculates the probability of observing this data and propagates the state with the highest probability of node 1650 to node 1680.

The nodes 1630 in turn comprise one or more nodes 1660 that correspond to the phenotype signature, expressed as geometric coordinates (i.e., the geometric coordinates in n-dimensional space of the clinical phenotype) and one or more nodes 1670 that relate to additional phenotype characteristics (e.g., quantitative measurements of a physiological characteristic). These genotype and phenotype nodes 1640 to 1670 in turn inform one or more child nodes 1680.

Node 1660 represents the projection of the phenotype observed in a patient or case with genetic disease in a n-dimensional geometric space, in which each list of phenotypes processed is identified by n-coordinates specifying its exact location in this n-dimensional space. This n-dimensional geometric space is an abstract representation of all phenotype signatures observed in patients or cases with genetic diseases, defined by semantically or clinically relevant relationships between them (e.g., as represented in Human Phenotype Ontology). This n-dimensional geometric space is obtained by performing metric-preserving dimensionality reduction (e.g., multi-dimensional scaling) to the matrix of similarity scores calculated for a complete list of phenotypes being processed. The connection between node 1660 and node 1680 represents the statistical dependency between the phenotype signature of a patient or case with genetic disease and a genetic disease potentially associated with this list of phenotypes. Node 1660 receives input data pertaining to the phenotype signature of a patient or case being processed. Based on this, node 1660 calculates the probability of observing this data and propagates the state with the highest probability of node 1660 to node 1680.

Node 1670 represents additional information pertaining to the phenotype being processed (such as confidence score for the classification, output from process 110). The connection between node 1670 and node 1680 represents the statistical dependency between additional information pertaining to the list of clinical phenotypes being processed and a genetic disease potentially associated with this list of phenotypes. Node 1670 receives input data pertaining to the list of clinical phenotypes being processed. Based on this, node 1670 calculates the probability of observing this data and propagates the state with the highest probability of node 1670 to node 1680.

As mentioned above, node 1680 represents genetic diseases, caused by one or more genetic variants and manifesting clinically with one or more clinical phenotypes. Node 1680 receives from nodes 1640, 1650, 1660 and 1670 the states with the highest probability for nodes 1640, 1650, 1660 and 1670 as calculated by each node. During “production,” node 1680 calculates the probability of a patient or case having a genetic disease based on the values from nodes 1640, 1650, 1660 and 1670, for each combination of genetic variant processed, phenotype observed in the patient and possible genetic disease known from the training data. Node 1680 outputs a list of probabilities predicted for each combination of genetic variant processed, phenotype observed in the patient and possible genetic disease known from the training data. Finally, this list is ranked in decreasing order on the value of the predicted probability (from most likely molecular and clinical diagnosis to least likely).

In embodiments the following data features are used to make graded predictions on the disease id in node 1680, given the observed genetic and phenotype data in an individual or clinical case:
Genotype information (hidden node 1620), deriving inputs from, for example:
   Coordinates from genotype signature for the genetic variant being evaluated in n-dimensional space (observed nodes 1640)
   Additional information relating to the classification of the genetic variant being evaluated, such as classification and/or confidence (observed nodes 1650)
Phenotype information (hidden node 1630), deriving inputs from, for example:
   Coordinates from phenotype signature being evaluated in n-dimensional space (observed nodes 1660)
   Additional information relating to the patient or case's clinical features, such as frequency of a specific observation or quantitative measurement of a clinical feature (observed nodes 1670).

Observed nodes 1640 take as input an n-tuple of coordinates corresponding to the genotype signature derived through multidimensional scaling. Observed nodes 1650 take as input data additional information relevant to the genotype such as the variant classification and/or the confidence scores in the prediction produced in step 110. Node 1620 is a hidden node that integrates information from node 1640 (genotype signature), node 1650 (additional information related to the genotype) and node 1680 (disease node).

Observed nodes 1660 take as input an n-tuple of coordinates corresponding to the phenotype signature derived through multidimensional scaling. Observed nodes 1670 take as input the data vector containing additional information relevant to the phenotype such as frequency of a specific observation or quantitative measurement of a clinical feature. Node 1630 is a hidden node that integrates information from node 1660 (phenotype signature), node 1670 (additional information related to the phenotype) and node 1680 (disease node).

The predictions of the states of individual nodes in the network 1600 are estimated by applying Bayes Rule to calculate the posterior predicted probabilities for any given node given the observed data, the model connectivity and the estimated parameters.

During training, the conditional model parameters (i.e., the parameters of the conditional probability distributions or CPDs) for each node in the network 1600 are estimated from the training dataset. Additionally, during training, the hidden nodes 1620 estimate the joint probability distribution of the genotype data input. Node 1640 in particular provides a probability of the genotype signature provided in a particular row of the input data shown in FIG. 17 provided through input node 1610 and node 1650 provides a probability of the classification of the genetic variant provided in a particular row of the input data shown in FIG. 17 provided through input node 1610. The hidden nodes 1630 estimate the joint probability distribution of the phenotype data input in node 1660 and 1670. Node 1660 in particular provides a probability of the phenotype signature provided in a particular row of the input data shown in FIG. 17 provided through input node 1610 and node 1670 provides a probability of the additional information relating to the patient or case's clinical features provided in a particular row of the input data shown in FIG. 17 provided through input node 1610.

In addition, during training, a known disease id is input in node 1680 directly from node 1610. It will be appreciated that, in “production mode,” this information is not available and that it is exactly this information that, in “production mode,” is being provided as output by node 1680.

The joint probability distributions provided by nodes 1620 and 1630 reflect the probability of simultaneously observing particular combinations of values over all observed nodes for which there is data available. During training, both hidden nodes 1620 and 1630 estimate the joint probability distribution of the genotype data input in node 1640 and 1650, the phenotype data input in node 1660 and 1670 and the disease ids input in node 1680. This entails that nodes 1620 and 1630 work in combination to estimate the joint probability distribution for any given combination of observations for the genotype, phenotype and disease present in the training data. Based on the predictions provided by nodes 1640 to 1670, node 1680 provides a disease id that has the highest predicted probability of applying to the predictions provided by nodes 1640 to 1670. Associated with each predicted probability output by node 1680 is an indication of confidence in the prediction. In one embodiment, predictions with a confidence score that is below a predetermined threshold are de-prioritized or not included in a prioritized list of genetic variants.

During training, the Expectation-Maximization (EM) algorithm is used to estimate the parameters of the conditional probability distributions for each node in network 1600. The EM algorithm allows to perform maximum likelihood estimation to find the values for the parameters that maximize the performance of the network to predict the observed data (i.e., disease id observed matching disease id predicted by nodes 1640 to 1680).

In the trained hierarchical Bayesian network for variant prioritization, the probabilities associated with the states of a variable reflect the knowledge extracted from training in the form of a-priori expectations (probabilities derived from the training data during training of the model and before providing input patient data).

During regular use of the trained network, node 1680 predicts the label of the genetic disease observed (i.e., disease id), given the genotype-phenotype combinations observed in a patient. In this case, the patient's genotype signature data is input in node 1610 and, from there, supplied to node 1640 and additional data pertaining to their genotype is input in node 1610 and, from there, supplied to node 1650. The phenotype signature data is input in node 1610 and supplied to node 1660 and additional data pertaining to their phenotype is input in node 1610 and supplied to node 1670. This data, together with the parameters of the probability distributions estimated during training of the model, is used by disease node 1680 to calculate the posterior probability of each disease label for all genotype-phenotype combinations observed in the patient and provided in the input data. In this process, trained nodes 1640 to 1670 and trained node 1680 perform the same function as described above when discussing the training phase of network 1600. The posterior probability of the disease labels predicted by node 1680 for all genotype-phenotype combinations is then used to sort all genotype-phenotype-disease combinations from highest to lowest posterior probability. The list of genotype-phenotype-disease combinations ranked by posterior probability in step 1830 shown in FIG. 20 and accompanied by their confidence score is provided as an output from the variant prioritization network.

Indications of the certainty of the model predictions for the disease label are calculated per single prediction (i.e., posterior probability for each predicted disease label and confidence score). Both the probability of the prediction and the confidence score are expressed as percentages. The confidence score for single predictions is calculated from the posterior probability of each prediction and the credible interval. The confidence score for single predictions indicates the likelihood of the predicted genetic disease label under individual combinations of genotype and phenotype data.

An example of the prioritization process is illustrated in FIG. 20. As is shown in this Figure, the prioritization algorithm can have two different phases, a first phase 1710 in which it is trained and a second phase 1720 in which the trained prioritization model is used to prioritize genotype and phenotype data in terms of their predicted association with a given genetic disease.

The training phase 1710 itself has two phases, an initial phase 1730 in which an initially untrained machine learning model is trained to enable it to prioritize correctly, and an ongoing training phase 1740 in which an already functional (and possibly also operational) model is continuously re-trained and the original training refined using newly available training data. In an embodiment, the same steps are performed during both training phases. The training is based on initial or new raw training data that is received in step 1750. From these data, genotype and phenotype signatures are extracted in step 1760 (as discussed above) before they are used in training the machine learning prioritizer in step 1770.

As discussed above, the hierarchical Bayesian network for variant prioritization is trained on genotype-phenotype-disease associations, consisting of lists of associations between a genetic disease and individual genotype and phenotype signatures. This training data is initially derived from curated datasets (e.g., databases such as ClinVar, DDG2P or Orphanet), composed of associations of classified disease-causing genetic variants (i.e., genotype), their associated clinical features (i.e., phenotype, presented for instance as a list of HPO terms) and their corresponding disease label (i.e., associated genetic disease, presented for instance as a OMIM disease id, if available).

It will be appreciated that differences in the preparation of signatures between training and regular use exist. Genotype signature extraction takes a genetic variant as input (e.g., a pathogenic variant causing a genetic disease in the training data 1750 or a candidate disease-causing genetic variant identified in an individual patient 1780). This genetic variant corresponds to the input vector in FIG. 18. Alternatively, genotype signature extraction can take a gene as input (e.g., a gene known to be associated with disease in the training data 1750 or a gene suspected to be affected by a disease-causing variant in an individual patient 1780).

During preparation of training data, genotype signature extraction is carried out on all individual genetic variants or genes in the training data.

During regular use of the trained algorithm for variant prioritization, the first step in genotype signature extraction, in which the terms are mapped back to the corresponding ontology, remains the same. In the second step (see FIG. 18), the similarity score is calculated for all possible pair-wise combinations between each individual genetic variant or gene newly submitted for the patient and all existing genetic variants or genes in the training data. This entails that every genetic variant submitted is mapped back to a coordinate system composed by all genetic variants or genes processed during the training process. The similarity between individual genes or genetic variants is computed as the semantic distance between terms in the corresponding ontology graph (e.g., gene ontology graph). A standard metric which incorporates graph information (e.g., shortest path length between two terms or minimum common ancestor) can be used for this purpose. In the third step (see FIG. 18), all pair-wise similarity scores obtained previously are organized in a multidimensional matrix of genotype similarities (i.e., representation similarity matrix). In the fourth and final step for genotype signature extraction when processing data for regular use of the trained network, dimensionality reduction via multidimensional scaling (MDS) is performed on this matrix to generate the principal components to be used subsequently in the prioritization step. Additionally, the principal components of the genotype signature can be visualized to provide diagnostic summary statistics useful to evaluate the quality of the data structures (e.g., signal to noise ratio, outliers).

The signature extraction pipeline is then used to extract the signatures for the genotype data 1780 and phenotype data 1800. The genotype data includes some or all genetic variants of a patient with classifications and their respective posterior probabilities. In an embodiment, these data are in the form of a standard VCF file as is in already use in the field, with the classifications and posterior probabilities as annotated columns. The trained model, based on the extracted genotype and phenotype signatures 1790 and 1810, characterizes the combinations of genotype, phenotype and diseases in step 1820 and outputs a list of disease-genetic variant-phenotype associations, together with the associated posterior probability of the prediction. The trained model takes into account all disease label predictions generated by the prioritization network (i.e., node 1680) for all combinations of genotype and phenotype data observed in the patient (including genotype and phenotype signatures, but also additional genotype and phenotype data such as variant classifications, posterior probabilities associated with the classifications which can be input in one or more nodes 1650 and both qualitative and quantitative physiological and/or phenotypic data which can be input in one or more nodes 1670).

The final step of variant prioritization 1830 is based on analyzing the probability that a particular combination of genotype and phenotype signatures is the cause of the target disease (i.e., how predictive is a combination of genotype and phenotype for a given disease). The posterior probabilities for all disease label-genotype-phenotype signature associations are ranked from the highest to the lowest, corresponding to a prioritization in terms of combined molecular and clinical relevance (e.g., from the most likely predictor of the molecular and clinical disease to the least likely). Depending on the focus of interest for the analysis (phenotype-, genetic- or disease-focused), different methods are used to rank the posterior probabilities (e.g., filtering the posterior probabilities by specific genes). Importantly, in one embodiment, when a disease label is not available (e.g., for example because the particular combination of phenotype and genotype information has either not been encountered before in relation to a known disease), a list of known phenotype-genotype associations from all known diseases which are most similar to the input genotype and phenotype data is returned.

After training, the hierarchical Bayesian network for variant prioritization can be used to prioritize a new set of clinical features and/or a new list of genetic variants in terms of their predictive power (e.g., associative strength) for any disease either known or unknown.

Three different embodiments are presented here for variant prioritization reflecting a phenotype-focused, genotype-focused or disease-focused prioritization approach (see below for further detail). In one embodiment, the obtained posterior probabilities are ranked first by variant and then by disease (from largest to smallest). This embodiment produces a list of variants prioritized for disease likelihood (i.e., genotype-focused prioritization).

In another embodiment, the obtained posterior probabilities are ranked first by phenotype and then by disease (from largest to smallest). This embodiment produces a list of phenotypes prioritized for disease likelihood (i.e., phenotype-focused prioritization).

In another embodiment, the obtained posterior probabilities are ranked by disease label (from largest to smallest). This embodiment produces a list of diseases prioritized for phenotype-genotype likelihood (i.e., disease-focused prioritization).

### Input Data for the Hierarchical Bayesian Network for Variant Prioritization

Candidate disease-causing genetic variants can be obtained by classifying a list of genetic variants identified in the patient's DNA sample either manually or using the hierarchical Bayesian network for variant classification described above. Alternatively, the list of candidate disease-causing genetic variants can be a list of unclassified genetic variants identified in the patient's DNA sample (i.e., genetic variants not having undergone genetic variant classification). Clinical features can be submitted in the ontology directly by the medical doctor, can be converted from a clinical description in natural language by a human analyst or can be converted into terms within a medical or clinical ontology, such as HPO terms, using natural language processing methods. Additionally, both qualitative and quantitative physiological and/or phenotypic data can be submitted as part of the terms describing the clinical abnormalities encountered in the patient.

While the list of genetic variants and clinical features (e.g., HPO terms) are processed into genotype and phenotype signatures and input as such into the hierarchical Bayesian network for variant classification, additional data vectors can be input in the model. Additional genotype data such as variant classifications or posterior probabilities associated with the classifications can be input in one or more nodes 1650 of the hierarchical Bayesian network for variant prioritization and included as part of the data analyzed by the prioritization network to generate predictions. Additional phenotype data such as both qualitative and quantitative physiological and/or phenotypic data identified in the patient can be provided as input in one or more nodes 1670 of the hierarchical Bayesian network for variant prioritization.

### Software Embodiments

The classification and prioritization models described in the previous sections can be instantiated in different ways. In one embodiment, the models are maintained and run in a cloud-based solution with the models running on a high-performance computing (HPC) cluster cloud based (e.g., AWS, Google) and interfacing with one or more data repositories (e.g., research institutes, private data storage sites, AWS). In another embodiment, the models can be instantiated locally in the form of a self-contained software. Additionally, the systems can be instantiated in a mix-and-match embodiment with different components being instantiated in different mediums (i.e., cloud-based solution for instantiating the ML models and a local-based solution for data repositories).

### Cloud Service

In one embodiment, the variant scientist is granted access to a user interface providing cloud-based services (annotation, classification, prioritization). The user interface provides a way to variant scientists in genetic testing labs of uploading VCF files and patient phenotypes for analysis by the above described classification and prioritization algorithms. Once submitted, the .vcf file and the .hpo file will be analyzed and results retrieved from the pre-computed database where available. If not available (e.g., a new mutation), upon approval of the user, ML services will be deployed on the cloud to analyze on-line the patient's data.

### Local Service

In this embodiment the algorithms are implemented in software which needs to be locally installed before use (e.g., on Windows, MacOSX or Linux operating systems). Once installed, the software will be able to perform all of the services on locally provided files completely on premise. All of the calculations are run on the local computer (e.g., PC) CPU and therefore computations performed by the local processor need access exclusively to the data stored in the local data storage facility. Interfacing of the machine learning model with the data is realized by the available APIs for the software program of choice.

Whilst certain embodiments have been described, these embodiments have been presented by way of example only and are not intended to limit the scope of the inventions. Indeed, the novel devices, and methods described herein may be embodied in a variety of other forms; furthermore, various omissions, substitutions and changes in the form of the devices, methods and products described herein may be made without departing from the spirit of the inventions. The accompanying claims and their equivalents are intended to cover such forms or modifications as would fall within the scope and spirit of the inventions.

1. A method of classifying a genetic variant executed on one or more processing resources comprising:
receiving, at a first plurality of trained nodes of a hierarchical Bayesian Network, input data comprising data of a genetic variant of a patient,
receiving, said input data at a second plurality of trained nodes of the hierarchical Bayesian Network;
receiving, at one or more trained nodes of the hierarchical Bayesian Network input from the first plurality of trained nodes and from the second plurality of trained nodes;
providing by the one or more nodes a posterior probability of a functional disruption the genetic variant causes; and
classifying the genetic variant using said posterior probability of the functional disruption caused by the genetic variant;
wherein the first plurality of nodes within the plurality of trained nodes are configured to represent a constraint of a genetic region to variation and wherein the second plurality of nodes within the plurality of trained nodes are configured to represent molecular consequences of a genetic variant.

2. A method as claimed in claim 1, wherein at least one of nodes of the first plurality of trained nodes or nodes of the second plurality of trained nodes are further configured to receive experimentally determined input data of at least one of the molecular consequences or the functional consequences of a genetic variant.

3. A method as claimed in claim 1, wherein said input data further comprise at least one of:
one or more individual genetic features of the genetic variant;
one or more biological features of the genetic variant; or
one or more clinical features of the genetic variant.

4. A method executed in on or more processing resources, the method comprising:
receiving input data comprising an indication of a biological relationship of a genetic variant of the patient with other genetic variants in a reference data set of known disease causing genetic variants at a first plurality of trained nodes of a hierarchical Bayesian Network;
using the first plurality of trained nodes to determine a likelihood of the biological relationship of the genetic variant with other genetic variants in the reference data set of known disease causing genetic variants occurring;
receive input data comprising a plurality of clinical features of the patient in relation to other pluralities of clinical features in a reference data set of known clinical features at a second plurality of trained nodes of a hierarchical Bayesian Network;
using the second plurality of trained nodes to determine a likelihood of the data comprising the plurality of clinical features of the patient occurring in relation to the reference data set of known clinical features;
at least one of:
attributing a disease label to the received input data; or
determining an indication of a likelihood of co-occurrence of the genetic variant of the patient and the plurality of clinical features of the patient;
using one or more further nodes based on input from the first plurality of trained nodes and from the second plurality of trained nodes.

5. A method as claimed in claim 4, wherein the one or more further nodes are configured to both attribute a disease label to the received input data and determine a likelihood of co-occurrence of the genetic variant of the patient and the phenotype of the patient, the one or more further nodes configured to:
receive a plurality of disease labels attributed to a plurality of genetic variants and a plurality of indications of the likelihood of the individual ones of the genetic variants co-occurring with the phenotype of the patient; and
rank the plurality of disease labels in order of their associated indications of likelihood of co-occurrence of the genetic variant with the phenotype of the patient.

6. A method of training a hierarchical Bayesian Network for classifying genetic variants comprising:
identifying one or more nodes that provide a low predictive quality within the hierarchical Bayesian Network;
using biological experiments to generate training data relating to the identified one or more nodes or relating to an observed node providing input to the identified one or more nodes; and
training said hierarchical Bayesian Network using said training data.

7. A method as claimed in claim 6, further comprising using said data generated using biological experiments to determine at least one of molecular scores or functional scores; and
training said model using said determined scores.


SEQUENCE LISTING (4 sequences)

SEQ ID NO: 1 - DNA, 47 bp, Homo sapiens
gattacagattacaagctgactgagtagctaggattacaggattaca

SEQ ID NO: 2 - DNA, 47 bp, Artificial Sequence
  Description of Artificial Sequence Synthetic oligonucleotide
gattacagattacacgctgactgagtagctaggattacaggattaca

SEQ ID NO: 3 - DNA, 47 bp, Artificial Sequence
  Description of Artificial Sequence Synthetic oligonucleotide
gattacagattacaggctgactgagtagctaggattacaggattaca

SEQ ID NO: 4 - DNA, 47 bp, Artificial Sequence
  Description of Artificial Sequence Synthetic oligonucleotide
gattacagattacatgctgactgagtagctaggattacaggattaca